(19) United States
(12) Patent Application Publication
(10) Pub. No.: US 2022/0213558 A1
(43) Pub. Date: Jul. 7, 2022

(54) METHODS AND SYSTEMS FOR URINE-BASED DETECTION OF UROLOGIC CONDITIONS

(71) Applicant: Convergent Genomics, Inc., South San Francisco, CA (US)

(72) Inventors: Trevor Gilpin Levin, South San Francisco, CA (US); Kevin Gregory Phillips, South San Francisco, CA (US); Mahdi Goudarzi, South San Francisco, CA (US)

(21) Appl. No.: 17/612,150

(22) PCT Filed: May 29, 2020
(86) PCT No.: PCT/US2020/035350

§ 371 Date: Nov. 17, 2021

Related U.S. Application Data
(60) Provisional application No. 62/872,439, filed on Jul. 10, 2019.
(60) Provisional application No. 62/855,261, filed on May 31, 2019.

(51) Int. Cl. C12Q 1/6886; G16B 20/20; G16B 40/20

(52) U.S. Cl. CPC C12Q 1/6886; G16B 20/20; C12Q 2600/112; C12Q 2600/156; G16B 40/20

(57) ABSTRACT

The present disclosure provides methods and systems directed to urine-based detection of urologic conditions. A method for identifying or monitoring a urologic condition of a subject may comprise processing a cell-free biological sample obtained or derived from the subject to generate a dataset indicative of a presence, absence, or relative assessment of the urologic condition; using a trained algorithm to process the dataset to determine a quantitative measure indicative of the presence, absence, or relative assessment of the urologic condition; based at least in part on the quantitative measure, identifying or providing an indication of the urologic condition with (i) a sensitivity of at least about 90%, (ii) a specificity of at least about 90%, (iii) a positive predictive value of at least about 90%, or (iv) a negative predictive value of at least about 90%; and electronically outputting a report that provides an indication of the urologic condition.

### CROSS-REFERENCE TO RELATED APPLICATIONS

This application claims benefit of priority under 35 U.S.C. § 119(e) of U.S. Ser. No. 62/855,261, filed May 31, 2019, and U.S. Ser. No. 62/872,439, filed Jul. 10, 2019, the entire content of both is incorporated herein by reference in its entirety

### STATEMENT OF GOVERNMENT SUPPORT

This invention was made with government support under Contract 4R44CA200174-02 awarded by the Department of Health and Human Services. The government has certain rights in the invention.

### FIELD OF THE INVENTION

The present invention relates generally to urologic conditions and more specifically to using machine learning and trained algorithms to provide an indication of the urologic status of a subject.

### BACKGROUND INFORMATION

Every year, about 80 thousand new cases of bladder cancer and about 18 thousand deaths from bladder cancer are reported in the U.S. Bladder cancer is the fourth most common cancer in men. Currently, urologic conditions such as bladder cancer can be diagnosed using clinical tests such as cystoscopy, biopsy, urine cytology, and imaging tests. However, widespread screening of asymptomatic adults for bladder cancer may be advantageous because five-year survival rates for bladder cancer are high if detected in its early stages. Thus, there exists a need for rapid, accurate screening methods for urologic conditions such as bladder cancer that are non-invasive and cost-effective.

### SUMMARY

The present disclosure provides methods, systems, and kits for detecting urologic conditions (e.g., bladder cancer, kidney cancer, and prostate cancer) by processing biological samples obtained from or derived from subjects. Cell-free or cell-associated biological samples (e.g., urine samples) obtained from subjects may be analyzed to measure a presence, absence, or relative assessment of the urologic condition (e.g., bladder cancer, kidney cancer, and prostate cancer). Such subjects may include subjects with a urologic condition and subjects without a urologic condition, e.g., a subject who may be at risk of developing a urologic condition.

In an aspect, the present disclosure provides a method for identifying or monitoring a urologic condition of a subject, comprising: (a) processing a biological sample obtained or derived from the subject to generate a dataset, wherein the dataset is indicative of a presence, absence, or relative assessment of the urologic condition of the subject; (b) using a trained algorithm to process the dataset to determine a quantitative measure indicative of the presence, absence, or relative assessment of the urologic condition of the subject; (c) based at least in part on the quantitative measure, identifying or providing an indication of the urologic condition of the subject with one or more of: (i) a sensitivity of at least about 90%, (ii) a specificity of at least about 90%, (iii) a positive predictive value of at least about 90%, and (iv) a negative predictive value of at least about 90%; and (d) electronically outputting a report that identifies or provides an indication of the urologic condition of the subject.

In some embodiments, the biological sample is urine or a derivative thereof. In some embodiments, the method further comprises processing a urine sample of the subject to obtain the biological sample. In some embodiments, processing the biological sample comprises polymerase chain reaction (PCR). In some embodiments, (c) comprises identifying or providing an indication of the urologic condition of the subject with two or more of: (i) a sensitivity of at least about 90%, (ii) a specificity of at least about 90%, (iii) a positive predictive value of at least about 90%, and (iv) a negative predictive value of at least about 90%. In some embodiments, (c) comprises identifying or providing an indication of the urologic condition of the subject with three or more of: (i) a sensitivity of at least about 90%, (ii) a specificity of at least about 90%, (iii) a positive predictive value of at least about 90%, and (iv) a negative predictive value of at least about 90%. In some embodiments, (c) comprises identifying or providing an indication of the urologic condition of the subject with (i) a sensitivity of at least about 90%, (ii) a specificity of at least about 90%, (iii) a positive predictive value of at least about 90%, and (iv) a negative predictive value of at least about 90%.

In some embodiments, (c) comprises identifying or providing an indication of the urologic condition of the subject with a sensitivity of at least about 90%. In some embodiments, (c) comprises identifying or providing an indication of the urologic condition of the subject with a sensitivity of at least about 95%. In some embodiments, (c) comprises identifying or providing an indication of the urologic condition of the subject with a sensitivity of at least about 99%. In some embodiments, (c) comprises identifying or providing an indication of the urologic condition of the subject with a specificity of at least about 90%. In some embodiments, (c) comprises identifying or providing an indication of the urologic condition of the subject with a specificity of at least about 95%. In some embodiments, (c) comprises identifying or providing an indication of the urologic condition of the subject with a specificity of at least about 99%. In some embodiments, (c) comprises identifying or providing an indication of the urologic condition of the subject with a positive predictive value (PPV) of at least about 90%. In some embodiments, (c) comprises identifying or providing an indication of the urologic condition of the subject with a PPV of at least about 95%. In some embodiments, (c) comprises identifying or providing an indication of the urologic condition of the subject with a PPV of at least about 99%. In some embodiments, (c) comprises identifying or providing an indication of the urologic condition of the subject with a negative predictive value (NPV) of at least about 90%. In some embodiments, (c) comprises identifying or providing an indication of the urologic condition of the subject with a NPV of at least about 95%. In some embodiments, (c) comprises identifying or providing an indication of the urologic condition of the subject with a NPV of at least about 99%. In some embodiments, (c) comprises identifying or providing an indication of the urologic condition of the subject with an Area Under Curve (AUC) of at least about 0.90. In some embodiments, (c) comprises identifying or providing an indication of the urologic condition of the subject with an Area Under Curve (AUC) of at least about 0.95. In some embodiments, (c) comprises identifying or providing an indication of the urologic condition of the subject with an Area Under Curve (AUC) of at least about 0.99.

In some embodiments, (a) comprises (i) subjecting the biological sample to conditions that are sufficient to isolate, enrich, or extract a plurality of nucleic acid molecules, and (ii) assaying the plurality of nucleic acid molecules to generate the dataset. In some embodiments, the method further comprises extracting a plurality of DNA molecules from the biological sample, and subjecting the plurality of DNA molecules to sequencing to generate a plurality of sequencing reads, wherein the dataset comprises the plurality of sequencing reads. In some embodiments, the sequencing is massively parallel sequencing. In some embodiments, the sequencing is performed at a depth of at least about 100-15,000×, at least about 100-10,000×, and more preferably at least about 100-5,000×. In some embodiments, the sequencing is performed at a depth of at least about 100-1000×. In some embodiments, the sequencing is performed at a depth of at least about 100-500×. In some embodiments, the sequencing comprises nucleic acid amplification. In some embodiments, the nucleic acid amplification comprises polymerase chain reaction (PCR). In some embodiments, the sequencing comprises use of simultaneous reverse transcription (RT) and polymerase chain reaction (PCR). In some embodiments, the method further comprises using probes configured to selectively enrich the plurality of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises at least 50,000 distinct genomic loci. In some embodiments, the panel of the one or more genomic loci comprises at least 100,000 distinct genomic loci.

In some embodiments, the method further comprises performing error suppression of the plurality of sequence reads by one or more of: (i) paired-end sequencing to correct sequencing errors, (ii) labeling and tracking of unique sequencing molecules within amplicons to suppress PCR and sequencing-induced errors, (iii) examining concordance of mutation calls on sense and antisense strands of the plurality of DNA molecules, (iv) suppression of noise profiles at the panel of one or more genomic loci using a plurality of reference cell-free and/or cell-associated biological DNA samples or (v) assessing the location of a putative single nucleotide variant position relative to the sequencing read cycle or location within the sequencing read and/or the location of the putative single nucleotide variant within the nucleic acid fragment and its proximity to the end of the fragment.

In some embodiments, the method further comprises performing error suppression of the plurality of sequence reads by two or more of: (i) paired-end sequencing to correct sequencing errors, (ii) labeling and tracking of unique sequencing molecules within amplicons to suppress PCR and sequencing-induced errors, (iii) examining concordance of mutation calls on sense and antisense strands of the plurality of DNA molecules, (iv) suppression of noise profiles at the panel of one or more genomic loci using a plurality of reference cell associated and/or cell-free biological DNA samples, or (v) assessing the location of a putative single nucleotide variant position relative to the sequencing read cycle or location within the sequencing read and/or the location of the putative single nucleotide variant within the nucleic acid fragment and its proximity to the end of the fragment. In some embodiments, the method further comprises performing error suppression of the plurality of sequence reads by three or more of: (i) paired-end sequencing to correct sequencing errors, (ii) labeling and tracking of unique sequencing molecules within amplicons to suppress PCR and sequencing-induced errors, (iii) examining concordance of mutation calls on sense and antisense strands of the plurality of DNA molecules, (iv) suppression of noise profiles at the panel of one or more genomic loci using a plurality of reference cell associated and/or cell-free biological DNA samples, or (v) assessing the location of a putative single nucleotide variant position relative to the sequencing read cycle or location within the sequencing read and/or the location of the putative single nucleotide variant within the nucleic acid fragment and its proximity to the end of the fragment. In some embodiments, the method further comprises performing error suppression of the plurality of sequence reads by (i) paired-end sequencing to correct sequencing errors, (ii) labeling and tracking of unique sequencing molecules within amplicons to suppress PCR and sequencing-induced errors, (iii) examining concordance of mutation calls on sense and antisense strands of the plurality of DNA molecules, (iv) suppression of noise profiles at the panel of one or more genomic loci using a plurality of reference cell associated and/or cell-free biological DNA samples, and (v) assessing the location of a putative single nucleotide variant position relative to the sequencing read cycle or location within the sequencing read and/or the location of the putative single nucleotide variant within the nucleic acid fragment and its proximity to the end of the fragment.

In one aspect, the method further includes using a machine learning algorithm trained to distinguish between falsely identified single nucleotide variants. For example, such variants may be produced due to sequencing errors or nucleic acid base specific damage (depurination or deamination) as opposed to being a true mutation registering as a positive signal.

In some embodiments, the biological sample is processed without nucleic acid isolation, enrichment, or extraction. In some embodiments, the report is presented on a graphical user interface of an electronic device of a user. In some embodiments, the user is the subject. In some embodiments, the method further comprises determining a likelihood of the identification or the indication of the urologic condition of the subject. In some embodiments, the subject is asymptomatic for the urologic condition.

In some embodiments, the trained algorithm is trained using a first set of independent training samples associated with presence of the urologic condition and a second set of independent training samples associated with absence of the urologic condition. In some embodiments, the method further comprises using the trained algorithm to process a set of clinical health data of the subject. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, the method further comprises providing the subject with a therapeutic intervention for the urologic condition. In some embodiments, the therapeutic intervention comprises surgery, chemotherapy, radiotherapy, immunotherapy, or a combination thereof. In some embodiments, the method further comprises monitoring the urologic condition, wherein the monitoring comprises assessing the urologic condition of the subject at a plurality of time points, wherein the assessing is based at least on the identification or the indication of urologic condition determined in (c) at each of the plurality of time points. In some embodiments, a difference in the assessment of the urologic condition of the subject among the plurality of time points is indicative of one or more clinical indications selected from the group consisting of: (i) a diagnosis of the urologic condition of the subject, (ii) a prognosis of the urologic condition of the subject, (iii) an efficacy or a non-efficacy of a course of treatment for treating the urologic condition of the subject, (iv) a resistance or a response of the urologic condition of the subject to a course of treatment for treating the urologic condition of the subject, and (v) a progression or a non-progression of the urologic condition of the subject.

In some embodiments, the urologic condition is selected from the group consisting of bladder cancer, kidney cancer, and prostate cancer. In some embodiments, the urologic condition is bladder cancer. In some embodiments, (b) comprises determining quantitative measures of one or more bladder cancer-associated genomic loci selected from: TP53, KDM6A, MLL2, ARID1A, PIK3CA, RHOA, CDKN2A, PPARG, ATM, TP53, PTEN, BEND3, and PLEKHS1. In some embodiments, the urologic condition is kidney cancer. In some embodiments, (b) comprises determining quantitative measures of one or more kidney cancer-associated genomic loci selected from: VHL, PBRM1, MUC, TTN, SETD1, RHOA, CDKN2A, PPARG, ATM, MLL2, TP53, SYNE1, LRP1B, and SETD2. In some embodiments, the urologic condition is prostate cancer. In some embodiments, (b) comprises determining quantitative measures of one or more prostate cancer-associated genomic loci selected from: ERG, TP53, MUC16, SPOP, SYNE1, PTEN, BEND3, ATM, MLL2, TP53, SYNE1, LRP1B, KDM6A, ARID1A, PIK3CA, FGFR3, and FOXA1. In some embodiments, the biological sample is a cell-free sample or a cellular sample. In one embodiment, the invention provides a method for determining a quantitative measure indicative of said presence, absence, or relative assessment of bladder cancer of said subject including determining quantitative measures of one or more bladder cancer-associated genomic loci selected from KDM6A, ARID1A, PIK3CA, FGFR3 and a combination thereof. Such genes are likely to be exclusive to bladder cancer as opposed to other urologic conditions. In one aspect, the method includes further determining quantitative measures of one or more bladder cancer-associated genomic loci selected from the group consisting of RHOA, CDKN2A, PPARG, ATM, MLL2, TP53, PTEN and BEND3. Such additional genes are believed to overlap between urologic conditions.

In another embodiment, the invention provides a method for determining a quantitative measure indicative of said presence, absence, or relative assessment of kidney cancer of said subject including determining quantitative measures of one or more kidney cancer-associated genomic loci selected from VHL, PBRM1, SETD2 and a combination thereof. Such genes are likely to be exclusive to kidney cancer as opposed to other urologic conditions. In one aspect, the method further includes determining quantitative measures of one or more kidney cancer-associated genomic loci selected from RHOA, CDKN2A, PPARG, ATM, MLL2, TP53, SYNE1 and LRP1B. Such additional genes are believed to overlap between urologic conditions.

In one embodiment, the invention provides a method for determining a quantitative measure indicative of said presence, absence, or relative assessment of prostate cancer of said subject including determining quantitative measures of one or more prostate cancer-associated genomic loci selected from ERG, SPOP, FOXA1 and a combination thereof. Such genes are likely to be exclusive to prostate cancer as opposed to other urologic conditions. In one aspect, the method further includes determining quantitative measures of one or more prostate cancer-associated genomic loci selected from PTEN, BEND3, ATM, MLL2, TP53, SYNE1 and LRP1B. Such additional genes are believed to overlap between urologic conditions.

In another embodiment, the invention provides a method for assessment or prediction of grade of a cancer. In one aspect, the grade of the cancer is assessed or predicted to be a high grade or low grade cancer. In another aspect, the grade of the cancer is assessed or predicted to be a Gleason score. In another aspect, the grade of the cancer is assessed or predicted as a 1-4 based on the Fuhrman system.

In another aspect, the present disclosure provides a computer system for identifying or monitoring a urologic condition of a subject, comprising: a database that is configured to store a dataset indicative of a presence, absence, or relative assessment of the urologic condition of the subject; and one or more computer processors operatively coupled to the database, wherein the one or more computer processors are individually collectively programmed to: (i) use a trained algorithm to process the dataset to determine a quantitative measure indicative of the presence, absence, or relative assessment of the urologic condition of the subject; (ii) based at least in part on the quantitative measure, identify or provide an indication of the urologic condition of the subject with one or more of: (i) a sensitivity of at least about 90%, (ii) a specificity of at least about 90%, (iii) a positive predictive value of at least about 90%, and (iv) a negative predictive value of at least about 90%; and (iii) electronically output a report that identifies or provides an indication of the urologic condition of the subject.

In some embodiments, the computer system further comprises an electronic display operatively coupled to the one or more computer processors, wherein the electronic display comprises a graphical user interface that is configured to display the report. In some embodiments, the urologic condition is selected from the group consisting of bladder cancer, kidney cancer, and prostate cancer.

In another aspect, the present disclosure provides a non-transitory computer readable medium comprising machine-executable code that, upon execution by one or more computer processors, implements a method for identifying or monitoring urologic condition of a subject, the method comprising: (a) processing a biological sample obtained or derived from the subject to generate a dataset, wherein the dataset is indicative of a presence, absence, or relative assessment of the urologic condition of the subject; (b) using a trained algorithm to process the dataset to determine a quantitative measure indicative of the presence, absence, or relative assessment of the urologic condition of the subject; (c) based at least in part on the quantitative measure, identifying or providing an indication of the urologic condition of the subject with one or more of: (i) a sensitivity of at least about 90%, (ii) a specificity of at least about 90%, (iii) a positive predictive value of at least about 90%, and (iv) a negative predictive value of at least about 90%; and (d) electronically outputting a report that identifies or provides an indication of the urologic condition of the subject.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example workflow of a method for urine-based detection of bladder cancer, in accordance with disclosed embodiments.

FIG. 2 illustrates development of a custom hybrid capture panel to analyze 140,000 bladder cancer disease loci. (A) Average number of unique genomes analyzed per sample. This level of library complexity allows a bladder cancer detection algorithm to implement multiple methods of noise suppression while confidently calling mutations at frequencies of as low as 1:1000 genomes. (B) Percent on target enrichment. Of all sequencing reads analyzed, over 80% are dedicated to our genes/loci of interest, and the remaining 20% “off-target” loci allow copy number variation algorithms to normalize hybrid capture performance within a sample. Compared to published technical performance of standard hybrid capture methods, this double capture approach achieves 30-40% higher on-target efficiency, achieving equivalent reductions in the amount of sequencing required (cost reduction). (C) Uniformity of coverage is achieved where greater than 96% of loci achieve coverage depth within 20% of the mean coverage. (D) Average sequencing depth. This high level of uniform coverage results in fewer low coverage loci and maximizes sensitivity across the panel. All values are average of 29 reference samples, error bars denote standard error of the mean.

FIG. 3 illustrates results showing that best-in-class mutation callers typically report a substantial number of false-positive mutations in deep sequencing of urine. Results from the Broad Institute's MuTect algorithm are reported for high-depth DNA sequencing of urine samples obtained from 15 healthy control subjects. Each column represents a gene, and each row represents a control urine sample. Young healthy controls are selected that have no history of cancer and with urine chemistries within normal range (no abnormalities in the 10 urine analytes measured). Selection of healthy normal urine is used to minimize the likelihood of true mutations and instead to illustrate the degree of false-positive mutation calls due to use of a mutation calling algorithm not optimized for the types of technical noise present in urine sequencing data. Each shaded box denotes a mutation called by MuTect in a gene (columns) and patient (rows), numbers in the boxes denote the number of events called within a gene where approximately half of positive samples have multiple false-positive mutations called within an individual gene. All control subjects are found to have one or more false-positive mutation calls. This data serves as a significant rationale for development of an improved diagnostic-grade mutation caller.

FIG. 4 illustrates results showing superior detection of tumor true positives in urine by UriSeq. The UriSeq and MuTect algorithms are used to define true-positive events in tumor DNA. The same algorithm is then used to detect the same mutational events in urine-derived DNA. The percentage of true positives detected quantifies the concordance of tumor variants and urine variants detected by the same algorithm. On average, UriSeq detected 77% of known true positives compared to only 41% by MuTect. UriSeq detected tumor signal in 100% of samples tested while MuTect failed to detect tumor signal in urine in 33% of samples. These two samples where MuTect failed on sensitivity were defined by lower allele frequency events. MuTect has been validated to call variants above 5% allele frequency.

FIG. 5 illustrates results showing that non-reference events are more prevalent in urine sequencing. In a 6-patient study, technical sequencing noise is investigated in paired peripheral blood, tumor, and urine samples collected from patients just prior to surgical removal of the tumor. The noise profile (defined as the number of non-reference events with alternate allele frequencies in our target detection range of 0.15% to 30%) is quantified. The mean number of loci contributing to noise across sample type is reported. Error bars denote standard error of the mean. These data demonstrate 34% more noise in urine-derived DNA compared to blood-derived DNA from the same individual, and 26% more noise in urine-derived DNA compared to tumor-derived DNA from the same individual.

FIGS. 6A-6B illustrates results showing that UriSeq noise suppression distinguishes noise from confident true-positive low-frequency mutations. FIGURE A Representative putative mutational profile (non-reference signal present in raw data) of urine-derived DNA and FIGURE B UriSeq algorithmic filtering (removal of noise signal) and identification of a high confidence mutational event (orange). These data are derived from a patient with bladder cancer and generated via analysis of matched tumor and urine samples. The vertical axis represents non-reference allele frequency, and the horizontal axis denotes genomic base pair location within the gene KDM6A. The detected signal in urine (Figure B), orange bar, is confirmed by a shared mutation signal found in sequencing the pure matched tumor. This mutation call is further supported as it was previously identified by The Cancer Genome Atlas (TCGA) Project and cBio Database as a hotspot loss of function mutation in other patients with bladder cancer. In this patient, the tumor signal is diluted by normal contaminating DNA in the urine such that the tumor signal intensity falls into the range of typical sequencing noise (Figure A). UriSeq's error suppression approach that (i) utilizes paired-end sequencing to correct sequencing errors, (ii) performs labeling and tracking of unique sequencing molecules within PCR duplicate copies to suppress both PCR and sequencing induced errors, (iii) utilizes the duplex nature of double-stranded DNA by defining read family strand information to quantify and mitigate DNA damage artifacts, and (iv) performs empirical modeling of noise profiles at each of the 140,000 loci using 25 reference urine DNA samples to remove all false-positive signal, leaving only the one true positive tumor matched event within KDM6A.

FIG. 7 illustrates results showing UriSeq variant detection algorithm sensitivity at various dilution levels. In response to the performance challenges observed in the MuTect algorithm, a urine-derived DNA optimized mutation caller is developed with extremely high specificity. In this experiment, 27 serial dilution samples are sequenced at high depth using known reference samples, and an algorithm is developed where stringency is set to eliminate all false positive calls. Among these 27 samples, 250 billion bases were analyzed with 0 false positive calls, establishing a specificity of less than 1 false positive per 250 billion bases analyzed. With this specificity, the presented sensitivity is achieved such that at a dilution where true-positive variants are present at 5% frequency, more than 94% of variants are correctly identified. When diluted to 1% frequency, more than 68% of variants are correctly identified. When diluted to 0.5%, more than 55% of variants are correctly identified.

FIG. 8 illustrates a computer system that is programmed or otherwise configured to implement methods provided herein.

FIGS. 9A and 9B illustrate a hybrid capture panel design strategy for urologic specificity, based on selection of gene panels for detection of bladder cancer, kidney cancer, and prostate cancer, which may comprise degenerate mutation genes and/or specific mutation genes, respectively.

FIG. 10 illustrates a “missing markers/quiet tumor” case in which both the number of CNVs and the number of mutations are low (left). Some samples have low mutation allele frequency (%) due to dilution (top right), and some samples have a low number of unique genomes due to fragmentation and low genome yield (bottom right).

FIG. 11 shows a graph illustrating a Model Training: Receiver Operating characteristic (ROC) curve for Bladder Cancer grade prediction. ROC was performed for calibrated Support Vector Machine (SVM) classifier using 10-fold cross validation. The problem was structured as a binary supervised learning with high grade tumor as positive label. In the ROC curve, the true positive rate (sensitivity) is plotted as a function of the false positive rate (1−specificity). The total number of subjects is 553 which 489 labeled high grade and 64 low grades. The area under the curve, AUC, is 0.89 which indicates the power of separability of the trained model.

FIG. 12 shows properties of the trained model: ranking the genes in prediction of BLCA grade. The final data consists of 553 subjects and 75 risk factors. The risk factors were engineered by combining mutated gene and amino acid changes either to missense or nonsense. 75 risk factors are ranked based on their contribution in the predictive power of the final classifier positively or negatively.

FIG. 13 shows a graph illustrating the Model Validation: Receiver Operating Characteristic (ROC) curve for Bladder Cancer grade prediction. After training our model (an ensemble support vector machine classifier) we explored the validity of the model on a cohort comprised of 35 individuals (LG=15, HG=20) whose urine-based DNA sequencing was inputted into the model. Grade was predicted with a sensitivity of 85% and specificity of 73%.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “subject,” generally refers to an entity or a medium that has testable or detectable genetic information. A subject can be a person or individual. A subject can be a vertebrate, such as, for example, a mammal. Non-limiting examples of mammals include murines, simians, humans, farm animals, sport animals, and pets.

The present disclosure provides methods, systems, and kits for detecting urologic conditions (e.g., bladder cancer, kidney cancer, and prostate cancer) by processing biological samples obtained from or derived from subjects. Cell-free biological samples (e.g., urine samples) or cellular samples obtained from subjects may be analyzed to measure a presence, absence, or relative assessment of the urologic condition (e.g., bladder cancer, kidney cancer, and prostate cancer). Such subjects may include subjects with a urologic condition and subjects without a urologic condition.

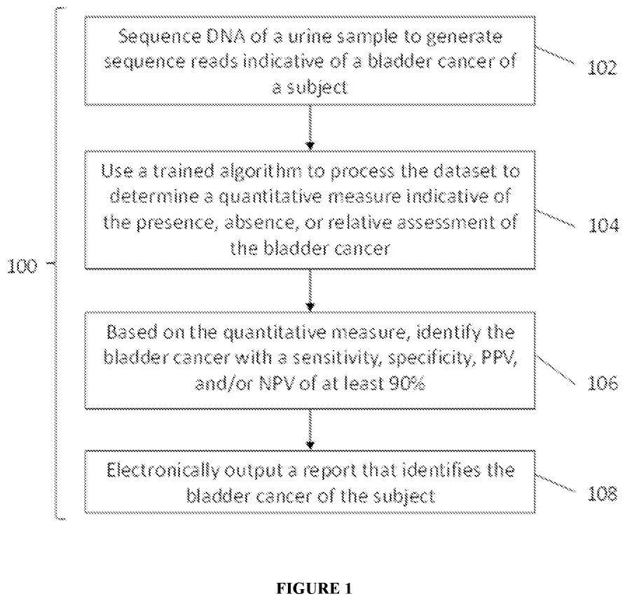

In some aspects, the present disclosure provides methods for urine-based detection of urologic conditions (e.g., bladder cancer, kidney cancer, and prostate cancer). For example, FIG. 1 illustrates an example workflow of a method for urine-based detection of bladder cancer, in accordance with disclosed embodiments. In an aspect, disclosed herein is a method 100 for identifying or monitoring bladder cancer in a subject. The method 100 may comprise processing a cell-free biological sample obtained or derived from the subject to generate a dataset indicative of a presence, absence, or relative assessment of the bladder cancer. For example, DNA of a urine sample may be sequenced to generate sequence reads indicative of a bladder cancer of a subject (as in operation 102). Next, a trained algorithm may be used to process the dataset to determine a quantitative measure indicative of the presence, absence, or relative assessment of the bladder cancer (as in operation 104). The trained algorithm may be configured to identify the bladder cancer with (i) a sensitivity of at least about 90%, (ii) a specificity of at least about 90%, (iii) a positive predictive value of at least about 90%, or (iv) a negative predictive value of at least about 90%. Next, based at least in part on the quantitative measure, an indication of the bladder cancer may be identified or provided with (i) a sensitivity of at least about 90%, (ii) a specificity of at least about 90%, (iii) a positive predictive value of at least about 90%, or (iv) a negative predictive value of at least about 90% (as in operation 106). A report may then be electronically outputted that identifies or provides an indication of the bladder cancer of the subject (as in operation 108).

### Processing Biological Samples

The biological samples may comprise cell-free or cellular biological samples, such as urine samples from a human subject. The cell-free or cellular samples may be stored in a variety of storage conditions before processing, such as different temperatures (e.g., at room temperature, under refrigeration or freezer conditions, at 4° C., at −18° C., −20° C., or at −80° C.) or different preservatives (e.g., alcohol, formaldehyde, or potassium dichromate).

The biological sample may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a lifestyle disease, an injury, a rare disease or an age related disease. The infectious disease may be caused by bacteria, viruses, fungi, and/or parasites. The cancer may be a urologic condition (e.g., bladder cancer, kidney cancer, and prostate cancer) or a urinary tract disease or disorder. The sample may be taken before and/or after treatment of a subject with a disease or disorder. Samples may be taken before and/or after a treatment. Samples may be taken during a treatment or a treatment regime. Multiple samples may be taken from a subject to monitor the effects of the treatment over time. The sample may be taken from a subject known or suspected of having a urologic condition (e.g., bladder cancer, kidney cancer, and prostate cancer) for which a definitive positive or negative diagnosis is not available via clinical tests.

The sample may be taken from a subject suspected of having a disease or a disorder. The sample may be taken from a subject experiencing unexplained symptoms, such as fatigue, nausea, weight loss, aches and pains, weakness, or memory loss. The sample may be taken from a subject having explained symptoms. The sample may be taken from a subject at risk of developing a disease or disorder due to factors such as familial history, age, environmental exposure, lifestyle risk factors, or presence of other known risk factors.

After obtaining a cell-free biological sample from the subject, the cell-free biological sample obtained from the subject may be processed to generate data indicative of a presence, absence, or relative assessment of a urologic condition (e.g., bladder cancer, kidney cancer, and prostate cancer) of a subject. For example, a presence, absence, or relative assessment of nucleic acid molecules of the cell-free biological sample at a panel of urologic condition-associated genomic loci (e.g., quantitative measures of mutations at a plurality of urologic condition-associated genomic loci) may be indicative of a urologic condition. Processing the biological sample obtained from the subject may comprise (i) subjecting the biological sample to conditions that are sufficient to isolate, enrich, or extract a plurality of nucleic acid molecules, and (ii) assaying the plurality of nucleic acid molecules to generate the dataset.

A plurality of nucleic acid molecules may be extracted from the cell-free biological sample and subjected to sequencing to generate a plurality of sequencing reads. The nucleic acid molecules may comprise deoxyribonucleic acid (DNA) or ribonucleic acid (RNA). The nucleic acid molecules (e.g., DNA or RNA) may be extracted from the cell-free biological sample by a variety of methods, such as a FastDNA Kit protocol from MP Biomedicals, a QIAamp DNA urine mini kit from Qiagen, or a urine DNA isolation kit protocol from Norgen Biotek. The extraction method may extract all DNA molecules from a sample. Alternatively, the extract method may selectively extract a portion of DNA molecules from a sample. Extracted RNA molecules from a sample may be converted to DNA molecules by reverse transcription (RT).

In some embodiments, both cell-free and cellular biological samples are obtained from the subject and analyzed. The cell-free and cellular biological samples may be separately obtained from the subject, or a biological sample containing a mixture of cell-free and cellular biological samples may be obtained from the subject. For example, a urine sample may contain both a cell-free fraction and a cellular fraction (e.g., bladder, kidney, or prostate tumor cells shed into the urine). As another example, a blood sample may contain both a cell-free fraction and a cellular fraction. In some embodiments, nucleic acids (e.g., DNA or RNA) are extracted from both the cell-free and cellular biological samples and sequenced, either separately or together, to produce a plurality of sequence reads. Algorithms may be used to identify sequence reads originating from each of the cell-free and the cellular biological samples.

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, and sequencing-by-hybridization, RNA-Seq (Illumina).

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., DNA) to a desired input quantity for subsequent sequencing. In some cases, the PCR may be used for global amplification of nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with one or more urologic conditions (e.g., bladder cancer, kidney cancer, and prostate cancer) (e.g., listed in databases such as TCGA or COSMIC). The genomic loci may comprise one or more of: single nucleotide variants (SNVs), copy number variants (CNVs), and insertions or deletions (indels). The genomic loci may be associated with a diagnosis, prognosis, resistance, recurrence of a urologic condition (e.g., bladder cancer, kidney cancer, and prostate cancer).

The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

In some embodiments, the biological samples may be assayed via a hybrid assay comprising both next-generation sequencing (NGS) and quantitative PCR (qPCR) to assess the urologic condition (e.g., bladder cancer, kidney cancer, and prostate cancer) of the subject. The NGS and qPCR assays may be performed using either the same or different panels of genomic loci (e.g., urologic condition-associated genomic loci). For example, a small panel of genes (e.g., TERT and PLEKHS1) which are specific to a urologic condition may be amenable to a qPCR assay.

DNA or RNA molecules may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each DNA molecule may be traced back to the sample (and the subject) from which the DNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the data indicative of the presence, absence, or relative assessment of the urologic condition. For example, the sequence reads may be aligned to one or more reference genomes (e.g., a genome of one or more species such as a human genome). The aligned sequence reads may be quantified at one or more genomic loci to generate the data indicative of a distribution of the presence, absence, or relative assessment of the urologic condition. For example, quantification of sequences corresponding to a plurality of genomic loci associated with a urologic condition may generate the data indicative of the presence, absence, or relative assessment of the urologic condition.

The urologic condition (e.g., bladder cancer, kidney cancer, and prostate cancer) may be identified or monitored in the subject by using probes configured to selectively enrich nucleic acid (e.g., DNA or RNA) molecules corresponding to the one or more genomic loci (e.g., urologic condition-associated genomic loci). The probes may be nucleic acid primers. The probes may have sequence complementarity with nucleic acid sequences from one or more of the individual genomic loci (e.g., urologic condition-associated genomic loci). The one or more genomic loci (e.g., urologic condition-associated genomic loci) may comprise at least about 2 thousand, at least about 3 thousand, at least about 4 thousand, at least about 5 thousand, at least about 6 thousand, at least about 7 thousand, at least about 8 thousand, at least about 9 thousand, at least about 10 thousand, at least about 11 thousand, at least about 12 thousand, at least about 13 thousand, at least about 14 thousand, at least about 15 thousand, at least about 16 thousand, at least about 17 thousand, at least about 18 thousand, at least about 19 thousand, at least about 20 thousand, at least about 40 thousand, at least about 60 thousand, at least about 80 thousand, at least about 100 thousand, at least about 120 thousand, at least about 140 thousand, at least about 160 thousand, at least about 180 thousand, at least about 200 thousand, or more distinct genomic loci (e.g., urologic condition-associated genomic loci).

The cell-free biological sample may be processed without any nucleic acid extraction. For example, the processing may comprise assaying the biological sample using probes that are selected for the one or more genomic loci (e.g., urologic condition-associated genomic loci). The one or more genomic loci (e.g., urologic condition-associated genomic loci) may comprise at least about 2 thousand, at least about 3 thousand, at least about 4 thousand, at least about 5 thousand, at least about 6 thousand, at least about 7 thousand, at least about 8 thousand, at least about 9 thousand, at least about 10 thousand, at least about 11 thousand, at least about 12 thousand, at least about 13 thousand, at least about 14 thousand, at least about 15 thousand, at least about 16 thousand, at least about 17 thousand, at least about 18 thousand, at least about 19 thousand, at least about 20 thousand, at least about 40 thousand, at least about 60 thousand, at least about 80 thousand, at least about 100 thousand, at least about 120 thousand, at least about 140 thousand, at least about 160 thousand, at least about 180 thousand, at least about 200 thousand, or more distinct genomic loci (e.g., urologic condition-associated genomic loci).

The probes may be nucleic acid molecules (e.g., DNA or RNA) having sequence complementarity with nucleic acid sequences (e.g., DNA or RNA) of the one or more genomic loci (e.g., urologic condition-associated genomic loci). These nucleic acid molecules may be primers or enrichment sequences. The assaying of the cell-free biological sample using probes that are selected for the one or more genomic loci (e.g., urologic condition-associated genomic loci) may comprise use of array hybridization, polymerase chain reaction (PCR), or nucleic acid sequencing (e.g., DNA sequencing or RNA sequencing).

The processing may comprise assaying the cell-free biological sample using probes that are selective for the one or more genomic loci (e.g., urologic condition-associated genomic loci) among other genomic loci in the cell-free biological sample. These probes may be nucleic acid molecules (e.g., DNA or RNA) having sequence complementarity with nucleic acid sequences (e.g., DNA or RNA) of the one or more genomic loci (e.g., urologic condition-associated genomic loci). These nucleic acid molecules may be primers or enrichment sequences. The assaying may comprise use of array hybridization, polymerase chain reaction (PCR), or nucleic acid sequencing (e.g., DNA sequencing or RNA sequencing).

The assay readouts may be quantified at one or more genomic loci (e.g., urologic condition-associated genomic loci) to generate the data indicative of a presence, absence, or relative assessment of the urologic condition. For example, quantification of array hybridization or polymerase chain reaction (PCR) corresponding to a plurality of genomic loci (e.g., urologic condition-associated genomic loci) may generate data indicative of a presence, absence, or relative assessment of the urologic condition. Assay readouts may comprise quantitative PCR (qPCR) values, digital PCR (dPCR) values, digital droplet PCR (ddPCR) values, fluorescence values, etc.

### Kits

Provided herein are kits for identifying or monitoring a urologic condition (e.g., bladder cancer, kidney cancer, and prostate cancer) in a subject. A kit may comprise probes for identifying a presence, absence, or relative amount of sequences at each of a plurality of urologic condition-associated genomic loci in a biological sample of the subject. A presence, absence, or relative amount of sequences at each of a plurality of urologic condition-associated genomic loci in the biological sample may be indicative of a urologic condition. The probes may be selective for the sequences at the plurality of urologic condition-associated genomic loci in the biological sample. A kit may comprise instructions for using the probes to process the biological sample to generate data indicative of a presence, absence, or relative amount of sequences at each of the plurality of urologic condition-associated genomic loci in a biological sample of the subject.

The probes in the kit may be selective for the sequences at the plurality of urologic condition-associated genomic loci in the biological sample. The probes in the kit may be configured to selectively enrich nucleic acid (e.g., DNA or RNA) molecules corresponding to the plurality of urologic condition-associated genomic loci. The probes in the kit may be nucleic acid primers. The probes in the kit may have sequence complementarity with nucleic acid sequences from one or more of the plurality of urologic condition-associated genomic loci. The plurality of urologic condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or greater different urologic condition-associated genomic loci.

The instructions in the kit may comprise instructions to assay the biological sample using the probes that are selective for the sequences at the plurality of urologic condition-associated genomic loci in the biological sample. These probes may be nucleic acid molecules (e.g., DNA or RNA) having sequence complementarity with nucleic acid sequences (e.g., DNA or RNA) from one or more of the plurality of urologic condition-associated genomic loci. These nucleic acid molecules may be primers or enrichment sequences. The instructions to assay the biological sample may comprise introductions to perform array hybridization, polymerase chain reaction (PCR), or nucleic acid sequencing (e.g., DNA sequencing or RNA sequencing) to process the biological sample to generate data indicative of a presence, absence, or relative amount of sequences at each of the plurality of urologic condition-associated genomic loci in the biological sample. A presence, absence, or relative amount of sequences at each of the plurality of urologic condition-associated genomic loci in the biological sample may be indicative of a urologic condition.

The instructions in the kit may comprise instructions to measure and interpret assay readouts, which may be quantified at one or more of the plurality of urologic condition-associated genomic loci to generate the data indicative of a presence, absence, or relative amount of sequences at each of the plurality of urologic condition-associated genomic loci in the biological sample. For example, quantification of array hybridization or polymerase chain reaction (PCR) corresponding to the plurality of urologic condition-associated genomic loci may generate data indicative of a presence, absence, or relative amount of sequences at each of the plurality of urologic condition-associated genomic loci in the biological sample. Assay readouts may comprise quantitative PCR (qPCR) values, digital PCR (dPCR) values, digital droplet PCR (ddPCR) values, fluorescence values, etc., or normalized values thereof.

### Trained Algorithms

After processing a biological sample from the subject, a trained algorithm may be used to process the data indicative of a presence, absence, or relative amount of sequences at each of the plurality of urologic condition-associated genomic loci to determine a presence, absence, or relative amount of sequences at each of the plurality of urologic condition-associated genomic loci in the biological sample. The trained algorithm may be configured to identify the urologic condition (e.g., bladder cancer, kidney cancer, and prostate cancer) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise data indicative of a presence, absence, or relative amount of sequences at each of the plurality of urologic condition-associated genomic loci. For example, an input variable may comprise a number of sequences corresponding to or aligning to each of the plurality of urologic condition-associated genomic loci.

The trained algorithm may comprise a classifier, such that each of the one or more output values comprises one of a fixed number of possible values (e.g., a linear classifier, a logistic regression classifier, etc.) indicating a classification of the biological sample by the classifier. The trained algorithm may comprise a binary classifier, such that each of the one or more output values comprises one of two values (e.g., {0, 1}, {positive, negative}, or {cancerous, non-cancerous}) indicating a classification of the biological sample by the classifier. The trained algorithm may be another type of classifier, such that each of the one or more output values comprises one of more than two values (e.g., {0, 1, 2}, {positive, negative, or indeterminate}, or {cancerous, non-cancerous, or indeterminate}) indicating a classification of the biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, cancerous, non-cancerous, or indeterminate. Such descriptive labels may provide an identification of a treatment for the subject's disease or disorder state, and may comprise, for example, a therapeutic intervention, a duration of the therapeutic intervention, and/or a dosage of the therapeutic intervention. Such descriptive labels may provide an identification of secondary clinical tests that may be appropriate to perform on the subject, and may comprise, for example, a biopsy, a blood test, a computed tomography (CT) scan, a magnetic resonance imaging (MRI) scan, an ultrasound scan, a chest X-ray, a positron emission tomography (PET) scan, or a PET-CT scan. Such descriptive labels may provide a prognosis of the disease or disorder state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may comprise, for example, an un-normalized probability value of at least 0. Such continuous output values may indicate a prognosis of the disease or disorder state of the subject and may comprise, for example, an indication of an expected or average progression-free survival (PFS) or overall survival (OS) of the subject. Such continuous output values may indicate a prediction of the course of treatment to treat the disease or disorder state of the subject and may comprise, for example, an indication of an expected duration of efficacy of the course of treatment. Some numerical values may be mapped to descriptive labels, for example, by mapping 1 to “positive” and 0 to “negative”.

Some of the output values may be assigned based on one or more cutoff values. For example, a binary classification of samples may assign an output value of “positive” or 1 if the sample indicates that the subject has at least a 50% probability of being diseased. For example, a binary classification of samples may assign an output value of “negative” or 0 if the sample indicates that the subject has less than a 50% probability of being diseased. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, and about 99%.

As another example, a classification of samples may assign an output value of “positive” or 1 if the sample indicates that the subject has a probability of being diseased of at least 50%, at least about 55%, at least about 60%, at least about 65%, at least about 70%, at least about 75%, at least about 80%, at least about 85%, at least about 90%, at least about 95%, at least about 98%, or at least about 99%. The classification of samples may assign an output value of “positive” or 1 if the sample indicates that the subject has a probability of being diseased of more than 50%, more than 55%, more than 60%, more than 65%, more than 70%, more than 75%, more than 80%, more than 85%, more than 90%, more than 95%, more than 98%, or more than 99%. The classification of samples may assign an output value of “negative” or 0 if the sample indicates that the subject has a probability of being diseased of less than 50%, less than 45%, less than 40%, less than 35%, less than 30%, less than 25%, less than 20%, less than 10%, less than 5%, less than 2%, or less than 1%. The classification of samples may assign an output value of “negative” or 0 if the sample indicates that the subject has a probability of being diseased of no more than 50%, no more than 45%, no more than 40%, no more than 35%, no more than 30%, no more than 25%, no more than 20%, no more than 10%, no more than 5%, no more than 2%, or no more than 1%. The classification of samples may assign an output value of “indeterminate” or 2 if the sample has not been classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a biological sample from a subject, associated data obtained by processing the biological sample (as described elsewhere herein), and one or more known output values corresponding to the biological sample (e.g., a clinical diagnosis, prognosis, treatment efficacy, or absence of a disease or disorder such as a urologic condition of the subject). Independent training samples may comprise biological samples and associated data and outputs obtained from a plurality of different subjects. Independent training samples may comprise biological samples and associated data and outputs obtained at a plurality of different time points from the same subject (e.g., before, after, and/or during a course of treatment to treat a disease or disorder of the subject). Independent training samples may be associated with presence of the urologic condition (e.g., bladder cancer, kidney cancer, and prostate cancer) (e.g., training samples comprising biological samples and associated data and outputs obtained from a plurality of subjects known to have the urologic condition). Independent training samples may be associated with absence of the urologic condition (e.g., bladder cancer, kidney cancer, and prostate cancer) (e.g., training samples comprising biological samples and associated data and outputs obtained from a plurality of subjects who are known to not have a previous diagnosis of the urologic condition, or otherwise who are asymptomatic for the urologic condition).

The trained algorithm may be trained with at least about 50, at least about 100, at least about 150, at least about 200, at least about 250, at least about 300, at least about 350, at least about 400, at least about 450, or at least about 500 independent training samples. The independent training samples may comprise samples associated with presence of the urologic condition and/or samples associated with absence of the urologic condition. The trained algorithm may be trained with no more than 500, no more than 450, no more than 400, no more than 350, no more than 300, no more than 250, no more than 200, no more than 150, no more than 100, or no more than 50 independent training samples associated with presence of the urologic condition. In some embodiments, the biological sample is independent of samples used to train the trained algorithm.

The trained algorithm may be trained with a first number of independent training samples associated with presence of the urologic condition and a second number of independent training samples associated with absence of the urologic condition. The first number of independent training samples associated with presence of the urologic condition may be no more than the second number of independent training samples associated with absence of the urologic condition. The first number of independent training samples associated with presence of the urologic condition may be equal to the second number of independent training samples associated with absence of the urologic condition. The first number of independent training samples associated with presence of the urologic condition may be greater than the second number of independent training samples associated with absence of the urologic condition.

The trained algorithm may be configured to identify the urologic condition (e.g., bladder cancer, kidney cancer, and prostate cancer) with an accuracy of at least about 90%, at least about 91%, at least about 92%, at least about 93%, at least about 94%, at least about 95%, at least about 96%, at least about 97%, at least about 98%, or at least about 99% for at least about 100 independent samples. The trained algorithm may be configured to identify the urologic condition with an accuracy of at least about 90%, at least about 91%, at least about 92%, at least about 93%, at least about 94%, at least about 95%, at least about 96%, at least about 97%, at least about 98%, or at least about 99% for at least about 150 independent samples. The trained algorithm may be configured to identify the urologic condition with an accuracy of at least about 90%, at least about 91%, at least about 92%, at least about 93%, at least about 94%, at least about 95%, at least about 96%, at least about 97%, at least about 98%, or at least about 99% for at least about 200 independent samples. The trained algorithm may be configured to identify the urologic condition with an accuracy of at least about 90%, at least about 91%, at least about 92%, at least about 93%, at least about 94%, at least about 95%, at least about 96%, at least about 97%, at least about 98%, or at least about 99% for at least about 250 independent samples. The trained algorithm may be configured to identify the urologic condition with an accuracy of at least about 90%, at least about 91%, at least about 92%, at least about 93%, at least about 94%, at least about 95%, at least about 96%, at least about 97%, at least about 98%, or at least about 99% for at least about 300 independent samples. The accuracy of identifying the urologic condition by the trained algorithm may be calculated as the percentage of independent test samples (e.g., subjects known to have the urologic condition or apparently healthy subjects with negative clinical test results for the urologic condition) that are correctly identified or classified as having or not having the urologic condition.

The trained algorithm may be configured to identify the urologic condition (e.g., bladder cancer, kidney cancer, and prostate cancer) with a positive predictive value (PPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The PPV of identifying the urologic condition by the trained algorithm may be calculated as the percentage of biological samples identified or classified as having the urologic condition that correspond to subjects that truly have the urologic condition. A PPV may also be referred to as a precision.

The trained algorithm may be configured to identify the urologic condition (e.g., bladder cancer, kidney cancer, and prostate cancer) with a negative predictive value (NPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The NPV of identifying the urologic condition by the trained algorithm may be calculated as the percentage of biological samples identified or classified as not having the urologic condition that correspond to subjects that truly do not have the urologic condition.

The trained algorithm may be configured to identify the urologic condition (e.g., bladder cancer, kidney cancer, and prostate cancer) with a clinical sensitivity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical sensitivity of identifying the urologic condition by the trained algorithm may be calculated as the percentage of independent test samples associated with presence of the urologic condition (e.g., subjects known to have the urologic condition) that are correctly identified or classified as having the urologic condition. A clinical sensitivity may also be referred to as a recall.

The trained algorithm may be configured to identify the urologic condition (e.g., bladder cancer, kidney cancer, and prostate cancer) with a clinical specificity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical specificity of identifying the urologic condition by the trained algorithm may be calculated as the percentage of independent test samples associated with absence of the urologic condition (e.g., apparently healthy subjects with negative clinical test results for the urologic condition) that are correctly identified or classified as not having the urologic condition.

The trained algorithm may be configured to identify the urologic condition (e.g., bladder cancer, kidney cancer, and prostate cancer) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. The AUC may be calculated as an integral of the Receiver Operator Characteristic (ROC) curve (e.g., the area under the ROC curve) associated with the trained algorithm in classifying biological samples as having or not having the urologic condition.

The trained algorithm may be adjusted or tuned to improve the performance, accuracy, PPV, NPV, clinical sensitivity, clinical specificity, or AUC of identifying the urologic condition (e.g., bladder cancer, kidney cancer, and prostate cancer). The trained algorithm may be adjusted or tuned by adjusting parameters of the trained algorithm (e.g., a set of cutoff values used to classify a sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of urologic condition-associated genomic loci may be identified as most influential or most important to be included for making high-quality classifications or identifications of urologic condition. The plurality of urologic condition-associated genomic loci or a subset thereof may be ranked based on metrics indicative of each genomic locus's influence or importance toward making high-quality classifications or identifications of urologic condition. Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, or AUC). For example, if training the training algorithm with a plurality comprising several dozen or hundreds of input variables in the trained algorithm results in an accuracy of classification of more than 99%, then training the training algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality results in decreased but still acceptable accuracy of classification (e.g., at least 90% or at least 95%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best metrics.

### Identifying or Monitoring a Urologic Condition

After using a trained algorithm to process the dataset indicative of the presence, absence, or relative assessment of the urologic condition (e.g., bladder cancer, kidney cancer, and prostate cancer), a quantitative measure indicative of the presence, absence, or relative assessment of the urologic condition may be determined, and the urologic condition may be identified or a progression or regression of the urologic condition may be monitored in the subject by identifying the subject as having the urologic condition with one or more of: (i) a sensitivity of at least about 90%, (ii) a specificity of at least about 90%, (iii) a positive predictive value of at least about 90%, and (iv) a negative predictive value of at least about 90%. The identification may be based at least in part on the presence, absence, or relative assessment of sequence reads of the dataset at a panel of urologic condition-associated genomic loci (e.g., quantitative measures of mutations at the urologic condition-associated genomic loci).

In some embodiments, the subject is assessed for a urologic condition (e.g., bladder cancer, kidney cancer, and prostate cancer) based on a referral as being at high risk for a urologic condition (e.g., based on a previous clinical or personal history), to determine a molecular grading of a urologic condition of the subject. For example, the subject may present with symptoms (e.g., visible blood in urine), personal history (e.g., age such as over 65 years old, or a smoking history), or clinical history (e.g., atypical cytology result) that indicates a high risk for a urologic condition (e.g., bladder cancer, kidney cancer, and prostate cancer). The assessment of the urologic condition of the subject may be performed to confirm a risk status (e.g., low risk or high risk) of the subject for the urologic condition, to determine a molecular grading of the urologic condition of the subject, and/or to select further testing or treatment options for the subject. For example, the subject may receive a recommendation for a secondary clinical test to confirm a diagnosis of the urologic condition (e.g., bladder cancer, kidney cancer, and prostate cancer). This secondary clinical test may comprise a cystoscopy, a biopsy, a urine cytology, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a reimbursement decision (e.g., for subsequent clinical tests, procedures, or treatment) may be made based on the molecular grading or risk assessment of the urologic condition of the subject. In some embodiments, a clinical decision (e.g., for subsequent clinical tests, procedures, or treatment) may be made based on the molecular grading or risk assessment of the urologic condition of the subject. For example, a determination of the risk that a surgery resection has a positive margin or the risk of mutations that are seeding recurrence may be made based on the molecular grading or risk assessment of the urologic condition of the subject. In some embodiments, a molecular sub-typing of the urologic condition may be made based on the molecular grading or risk assessment of the urologic condition of the subject. For example, a carcinoma in situ (a relatively aggressive form of cancer) may be identified (e.g., using a panel of genes correlated with carcinoma in situ).

In some embodiments, using methods and systems of the present disclosure, screening tests can be performed for a large population of subjects (e.g., all subjects of a certain age range or having certain personal or family history indicative of an elevated risk of one or more urologic conditions), toward initial diagnosis or early detection applications. In some embodiments, using methods and systems of the present disclosure, triage of patients can be performed for those patients presenting with symptoms (e.g., hematuria) which are indicative of one or more urologic conditions. In some embodiments, using methods and systems of the present disclosure, surveillance or monitoring of a patient for one or more urologic conditions can be performed to (i) quantify minimal residual disease (MRD) following standard of care (e.g., surgery) and/or to (ii) guide scoping intervals utilized by urologists to visually inspect organs or tissues (e.g., the bladder) using standard invasive scoping procedures. In some embodiments, using methods and systems of the present disclosure, an assessment of a subject for one or more urologic conditions can be performed to resolve atypical or indeterminate test results (e.g., cytology).

The urologic condition (e.g., bladder cancer, kidney cancer, and prostate cancer) may be identified in the subject with an accuracy of at least 90%, at least 91%, at least 92%, at least 93%, at least 94%, at least 95%, at least 96%, at least 97%, at least 98%, or at least 99%. The accuracy of identifying the urologic condition by the trained algorithm may be calculated as the percentage of independent test samples (e.g., subjects known to have the urologic condition or apparently healthy subjects with negative clinical test results for the urologic condition) that are correctly identified or classified as having or not having the urologic condition.

The urologic condition (e.g., bladder cancer, kidney cancer, and prostate cancer) may be identified in the subject with a positive predictive value (PPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The PPV of identifying the urologic condition by the trained algorithm may be calculated as the percentage of biological samples identified or classified as having the urologic condition that correspond to subjects that truly have the urologic condition. A PPV may also be referred to as a precision.

The urologic condition (e.g., bladder cancer, kidney cancer, and prostate cancer) may be identified in the subject with a negative predictive value (NPV) of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The NPV of identifying the urologic condition by the trained algorithm may be calculated as the percentage of biological samples identified or classified as not having the urologic condition that correspond to subjects that truly do not have the urologic condition.

The urologic condition (e.g., bladder cancer, kidney cancer, and prostate cancer) may be identified in the subject with a clinical sensitivity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical sensitivity of identifying the urologic condition by the trained algorithm may be calculated as the percentage of independent test samples associated with presence of the urologic condition (e.g., subjects known to have the urologic condition) that are correctly identified or classified as having the urologic condition. A clinical sensitivity may also be referred to as a recall.

The urologic condition (e.g., bladder cancer, kidney cancer, and prostate cancer) may be identified in the subject with a clinical specificity of at least about 5%, at least about 10%, at least about 15%, at least about 20%, at least about 25%, at least about 30%, at least about 35%, at least about 40%, at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. The clinical specificity of identifying the urologic condition by the trained algorithm may be calculated as the percentage of independent test samples associated with absence of the urologic condition (e.g., apparently healthy subjects with negative clinical test results for the urologic condition) that are correctly identified or classified as not having the urologic condition.

After the urologic condition (e.g., bladder cancer, kidney cancer, and prostate cancer) is identified in a subject, a stage of the urologic condition (e.g., stage I, stage II, stage III, or stage IV) may further be identified. The stage of the urologic condition may be determined based at least in part on the presence, absence, or relative assessment of sequence reads of the dataset at a panel of urologic condition-associated genomic loci (e.g., quantitative measures of mutations at the urologic condition-associated genomic loci).

Upon identifying the subject as having the urologic condition (e.g., bladder cancer, kidney cancer, and prostate cancer), the subject may be provided with a therapeutic intervention (e.g., prescribing an appropriate course of treatment to treat the urologic condition of the subject). The therapeutic intervention may comprise a surgical tumor resection, an effective dose of chemotherapy, an effective dose of radiotherapy, an effective dose of targeted therapy, an effective dose of immunotherapy. If the subject is currently being treated for the urologic condition with a course of treatment, the therapeutic intervention may comprise a subsequent different course of treatment (e.g., to increase treatment efficacy due to tumor resistance, tumor recurrence, non-response of the current course of treatment).

The therapeutic intervention may comprise recommending the subject for a secondary clinical test to confirm a diagnosis of the urologic condition (e.g., bladder cancer, kidney cancer, and prostate cancer). This secondary clinical test may comprise a cystoscopy, a biopsy, a urine cytology, an imaging test, a blood test, a computed tomography (CT) scan, a magnetic resonance imaging (MRI) scan, an ultrasound scan, a chest X-ray, a positron emission tomography (PET) scan, a PET-CT scan, PSA test or any combination thereof.

The subject may be treated upon identifying the subject as having the urologic condition (e.g., bladder cancer, kidney cancer, and prostate cancer). Treating the subject may comprise administering an appropriate therapeutic intervention to treat the urologic condition of the subject. The therapeutic intervention may comprise a surgical tumor resection, an effective dose of chemotherapy, an effective dose of radiotherapy, an effective dose of targeted therapy, an effective dose of immunotherapy. If the subject is currently being treated for the urologic condition with a course of treatment, the administered therapeutic intervention may comprise a subsequent different course of treatment (e.g., to increase treatment efficacy due to tumor resistance, tumor recurrence, non-response of the current course of treatment).

The presence, absence, or relative assessment of sequence reads of the dataset at the panel of urologic condition-associated genomic loci (e.g., quantitative measures of mutations at the urologic condition-associated genomic loci) may be assessed over a duration of time to monitor a patient (e.g., subject who has urologic condition or who is being treated for urologic condition). In such cases, the quantitative measures of mutations at the urologic condition-associated genomic loci of the patient may change during the course of treatment. For example, the quantitative measures of mutations at the urologic condition-associated genomic loci of a patient whose urologic condition is regressing due to an effective treatment (e.g., chemotherapy or surgical resection) may shift toward the profile or distribution of a healthy subject. Conversely, for example, the quantitative measures of mutations at the urologic condition-associated genomic loci of a patient whose urologic condition is progressing due to an ineffective treatment (e.g., when the tumor becomes resistant) may shift toward the profile or distribution of a subject with more advanced stage urologic condition.

The progression or regression of the urologic condition (e.g., bladder cancer, kidney cancer, and prostate cancer) in the subject may be monitored by monitoring a course of treatment for treating the urologic condition in the subject. The monitoring may comprise assessing the urologic condition in the subject at two or more time points. The assessing may be based at least on the presence, absence, or relative assessment of sequence reads of the dataset at a panel of urologic condition-associated genomic loci (e.g., quantitative measures of mutations at the urologic condition-associated genomic loci) determined at each of the two or more time points.

A difference in the presence, absence, or relative assessment of sequence reads of the dataset at a panel of urologic condition-associated genomic loci (e.g., quantitative measures of mutations at the urologic condition-associated genomic loci) determined between the two or more time points may be indicative of one or more clinical indications, such as (i) a diagnosis of the urologic condition in the subject, (ii) a prognosis of the urologic condition in the subject, (iii) a progression of the urologic condition in the subject, (iv) a regression of the urologic condition in the subject, (v) an efficacy of the course of treatment for treating the urologic condition in the subject, and (vi) a resistance of the urologic condition toward the course of treatment for treating the urologic condition in the subject.

A difference in the presence, absence, or relative assessment of sequence reads of the dataset at a panel of urologic condition-associated genomic loci (e.g., quantitative measures of mutations at the urologic condition-associated genomic loci) determined between the two or more time points may be indicative of a diagnosis of the urologic condition in the subject. For example, if the urologic condition was not detected in the subject at an earlier time point but was detected in the subject at a later time point, then the difference is indicative of a diagnosis of the urologic condition in the subject. A clinical action or decision may be made based on this indication of diagnosis of the urologic condition in the subject, e.g., prescribing a new therapeutic intervention for the subject.

A difference in the presence, absence, or relative assessment of sequence reads of the dataset at a panel of urologic condition-associated genomic loci (e.g., quantitative measures of mutations at the urologic condition-associated genomic loci) determined between the two or more time points may be indicative of a prognosis of the urologic condition in the subject.

A difference in the presence, absence, or relative assessment of sequence reads of the dataset at a panel of urologic condition-associated genomic loci (e.g., quantitative measures of mutations at the urologic condition-associated genomic loci) determined between the two or more time points may be indicative of a progression of the urologic condition in the subject. For example, if the urologic condition was detected in the subject both at an earlier time point and at a later time point, and if the difference is a negative difference (e.g., the presence, absence, or relative assessment of sequence reads of the dataset at a panel of urologic condition-associated genomic loci (e.g., quantitative measures of mutations at the urologic condition-associated genomic loci) increased from the earlier time point to the later time point), then the difference may be indicative of a progression (e.g., increased tumor load, tumor burden, or tumor size) of the urologic condition in the subject. A clinical action or decision may be made based on this indication of the progression, e.g., prescribing a new therapeutic intervention or switching therapeutic interventions (e.g., ending a current treatment and prescribing a new treatment) for the subject.

A difference in the presence, absence, or relative assessment of sequence reads of the dataset at a panel of urologic condition-associated genomic loci (e.g., quantitative measures of mutations at the urologic condition-associated genomic loci) determined between the two or more time points may be indicative of a regression of the urologic condition in the subject. For example, if the urologic condition was detected in the subject both at an earlier time point and at a later time point, and if the difference is a positive difference (e.g., the presence, absence, or relative assessment of sequence reads of the dataset at a panel of urologic condition-associated genomic loci (e.g., quantitative measures of mutations at the urologic condition-associated genomic loci) decreased from the earlier time point to the later time point), then the difference may be indicative of a regression (e.g., decreased tumor load, tumor burden, or tumor size) of the urologic condition in the subject. A clinical action or decision may be made based on this indication of the regression, e.g., continuing or ending a current therapeutic intervention for the subject.

A difference in the presence, absence, or relative assessment of sequence reads of the dataset at a panel of urologic condition-associated genomic loci (e.g., quantitative measures of mutations at the urologic condition-associated genomic loci) determined between the two or more time points may be indicative of an efficacy of the course of treatment for treating the urologic condition in the subject. For example, if the urologic condition was detected in the subject at an earlier time point but was not detected in the subject at a later time point, then the difference may be indicative of an efficacy of the course of treatment for treating the urologic condition in the subject. A clinical action or decision may be made based on this indication of the efficacy of the course of treatment for treating the urologic condition in the subject, e.g., continuing or ending a current therapeutic intervention for the subject.

A difference in the presence, absence, or relative assessment of sequence reads of the dataset at a panel of urologic condition-associated genomic loci (e.g., quantitative measures of mutations at the urologic condition-associated genomic loci) determined between the two or more time points may be indicative of a resistance of the urologic condition toward the course of treatment for treating the urologic condition in the subject. For example, if the urologic condition was detected in the subject both at an earlier time point and at a later time point, and if the difference is a negative or zero difference (e.g., the presence, absence, or relative assessment of sequence reads of the dataset at a panel of urologic condition-associated genomic loci (e.g., quantitative measures of mutations at the urologic condition-associated genomic loci) increased or remained at a constant level from the earlier time point to the later time point), and if an efficacious treatment was indicated at an earlier time point, then the difference may be indicative of a resistance (e.g., increased or constant tumor load, tumor burden, or tumor size) of the course of treatment for treating the urologic condition in the subject. A clinical action or decision may be made based on this indication of the resistance of the course of treatment for treating the urologic condition in the subject, e.g., ending a current therapeutic intervention and/or switching to (e.g., prescribing) a different new therapeutic intervention for the subject.

In some embodiments, the monitoring of the subject is informed by a previous clinical history of the subject, such as an initial or previous diagnosis of the subject for a urologic condition (e.g., a disease burden obtained from tumor analysis). For example, longitudinal monitoring of the subject can comprise performing a first classification algorithm that differentially weights or thresholds particular genes within a panel of genes which are previously seen as higher confidence and more informative (e.g., by decreasing sensitivity thresholds for those particular genes in longitudinal time course). As another example, longitudinal monitoring of the subject can comprise performing a second classification algorithm for cases where a patient presents with a recurrent tumor or is in the middle of surveillance protocol and does not have an initial or previous clinical history (e.g., initial diagnosis) of the urologic condition.

In some embodiments, the urologic condition is selected from bladder cancer, kidney cancer, and prostate cancer. In some embodiments, the urologic condition is bladder cancer. In some embodiments, (b) includes determining quantitative measures of one or more bladder cancer-associated genomic loci selected from: TP53, KDM6A, MLL2, ARID1A, PIK3CA, RHOA, CDKN2A, PPARG, ATM, TP53, PTEN, BEND3, and PLEKHS1 and a combination thereof.

In some embodiments, the urologic condition is kidney cancer. In some embodiments, (b) includes determining quantitative measures of one or more kidney cancer-associated genomic loci selected from VHL, PBRM1, MUC, TTN, SETD1, RHOA, CDKN2A, PPARG, ATM, MLL2, TP53, SYNE1, LRP1B, and SETD2 and a combination thereof.

In some embodiments, the urologic condition is prostate cancer. In some embodiments, (b) includes determining quantitative measures of one or more prostate cancer-associated genomic loci selected from ERG, TP53, MUC16, SPOP, SYNE1, PTEN, BEND3, ATM, MLL2, TP53, SYNE1, LRP1B, KDM6A, ARID1A, PIK3CA, FGFR3, and FOXA1 and a combination thereof.

In some embodiments, the biological sample is a cell-free sample or a cellular sample.

In one embodiment, the invention provides a method for determining a quantitative measure indicative of said presence, absence, or relative assessment of bladder cancer of said subject including determining quantitative measures of one or more bladder cancer-associated genomic loci selected from KDM6A, ARID1A, PIK3CA, FGFR3 and a combination thereof.

Such genes are likely to be exclusive to bladder cancer as opposed to other urologic conditions. In one aspect, the method includes further determining quantitative measures of one or more bladder cancer-associated genomic loci selected from the group consisting of RHOA, CDKN2A, PPARG, ATM, MLL2, TP53, PTEN and BEND3. Such additional genes are believed to overlap between urologic conditions.

In another embodiment, the invention provides a method for determining a quantitative measure indicative of said presence, absence, or relative assessment of kidney cancer of said subject including determining quantitative measures of one or more kidney cancer-associated genomic loci selected from VHL, PBRM1, SETD2 and a combination thereof. Such genes are likely to be exclusive to kidney cancer as opposed to other urologic conditions. In one aspect, the method further includes determining quantitative measures of one or more kidney cancer-associated genomic loci selected from RHOA, CDKN2A, PPARG, ATM, MLL2, TP53, SYNE1 and LRP1B. Such additional genes are believed to overlap between urologic conditions.

In one embodiment, the invention provides a method for determining a quantitative measure indicative of said presence, absence, or relative assessment of prostate cancer of said subject including determining quantitative measures of one or more prostate cancer-associated genomic loci selected from ERG, SPOP, FOXA1 and a combination thereof. Such genes are likely to be exclusive to prostate cancer as opposed to other urologic conditions. In one aspect, the method further includes determining quantitative measures of one or more prostate cancer-associated genomic loci selected from PTEN, BEND3, ATM, MLL2, TP53, SYNE1 and LRP1B. Such additional genes are believed to overlap between urologic conditions.

In another embodiment, the invention provides a method for assessment or prediction of grade of a cancer. In one aspect, the grade of the cancer is assessed or predicted to be a high grade or low grade cancer. In another aspect, the grade of the cancer is assessed or predicted to be a Gleason score. In another aspect, the grade of the cancer is assessed or predicted as a 1-4 based on the Fuhrman system.

### Outputting a Report of the Urologic Condition

After the urologic condition (e.g., bladder cancer, kidney cancer, and prostate cancer) is identified or a progression or regression of the urologic condition is monitored in the subject, a report may be electronically outputted that identifies or provides an indication of the progression or regression of the urologic condition in the subject. The subject may not display a urologic condition (e.g., is asymptomatic of the urologic condition). The report may be presented on a graphical user interface (GUI) of an electronic device of a user. The user may be the subject, a caretaker, a physician, a nurse, or another health care worker.

The report may include one or more clinical indications such as (i) a diagnosis of the urologic condition in the subject, (ii) a prognosis of the urologic condition in the subject, (iii) a progression of the urologic condition in the subject, (iv) a regression of the urologic condition in the subject, (v) an efficacy of the course of treatment for treating the urologic condition in the subject, and (vi) a resistance of the urologic condition toward the course of treatment for treating the urologic condition in the subject. The report may include one or more clinical actions or decisions made based on these one or more clinical indications.

For example, a clinical indication of a diagnosis of the urologic condition (e.g., bladder cancer, kidney cancer, and prostate cancer) in the subject may be accompanied with a clinical action of prescribing a new therapeutic intervention for the subject. As another example, a clinical indication of a progression of the urologic condition in the subject may be accompanied with a clinical action of prescribing a new therapeutic intervention or switching therapeutic interventions (e.g., ending a current treatment and prescribing a new treatment) for the subject. As another example, a clinical indication of a regression of the urologic condition in the subject may be accompanied with a clinical action of continuing or ending a current therapeutic intervention for the subject. As another example, a clinical indication of an efficacy of the course of treatment for treating the urologic condition in the subject may be accompanied with a clinical action of continuing or ending a current therapeutic intervention for the subject. As another example, a clinical indication of a resistance of the course of treatment for treating the urologic condition in the subject may be accompanied with a clinical action of ending a current therapeutic intervention and/or switching to (e.g., prescribing) a different new therapeutic intervention for the subject.

### Computer Systems

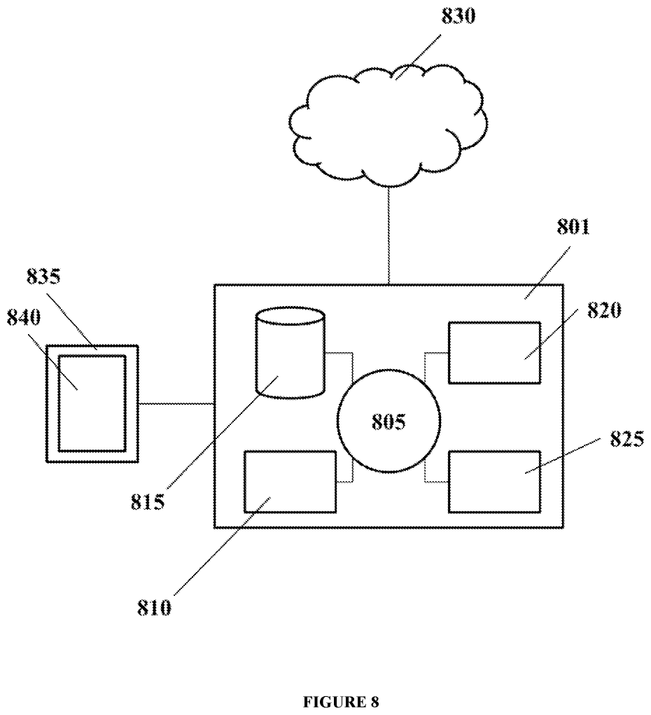

The present disclosure provides computer systems that are programmed to implement methods of the disclosure. FIG. 8 shows a computer system 801 that is programmed or otherwise configured to, for example, (i) train and test a trained algorithm, (ii) use the trained algorithm to process data indicative of a presence, absence, or relative assessment of urologic condition (e.g., bladder cancer, kidney cancer, and prostate cancer) of a subject, (iii) determine a quantitative measure indicative of a presence, absence, or relative assessment of urologic condition of a subject, (iv) identify or provide an indication of the urologic condition of the subject, or (v) electronically output a report that identifies or provides an indication of the urologic condition of the subject.

The computer system 801 can regulate various aspects of analysis, calculation, and generation of the present disclosure, such as, for example, (i) training and testing a trained algorithm, (ii) using the trained algorithm to process data indicative of a presence, absence, or relative assessment of urologic condition (e.g., bladder cancer, kidney cancer, and prostate cancer) of a subject, (iii) determining a quantitative measure indicative of a presence, absence, or relative assessment of urologic condition of a subject, (iv) identifying or providing an indication of the urologic condition of the subject, or (v) electronically outputting a report that identifies or provides an indication of the urologic condition of the subject. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet.

The network 830 in some cases is a telecommunication and/or data network. The network 830 can include one or more computer servers, which can enable distributed computing, such as cloud computing. For example, one or more computer servers may enable cloud computing over the network 830 (“the cloud”) to perform various aspects of analysis, calculation, and generation of the present disclosure, such as, for example, (i) training and testing a trained algorithm, (ii) using the trained algorithm to process data indicative of a presence, absence, or relative assessment of urologic condition (e.g., bladder cancer, kidney cancer, and prostate cancer) of a subject, (iii) determining a quantitative measure indicative of a presence, absence, or relative assessment of urologic condition of a subject, (iv) identifying or providing an indication of the urologic condition of the subject, or (v) electronically outputting a report that identifies or provides an indication of the urologic condition of the subject. Such cloud computing may be provided by cloud computing platforms such as, for example, Amazon Web Services (AWS), Microsoft Azure, Google Cloud Platform, and IBM cloud. The network 830, in some cases with the aid of the computer system 801, can implement a peer-to-peer network, which may enable devices coupled to the computer system 801 to behave as a client or a server.

The CPU 805 may comprise one or more computer processors and/or one or more graphics processing units (GPUs). The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, (i) a visual display indicative of training and testing of a trained algorithm, (ii) a visual display of data indicative of a presence, absence, or relative assessment of urologic condition (e.g., bladder cancer, kidney cancer, and prostate cancer) of a subject, (iii) a determined presence, absence, or relative assessment of urologic condition of a subject, (iv) an identification of a subject as having urologic condition, or (v) an electronic report that identifies or provides an indication of the urologic condition of the subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, (i) train and test a trained algorithm, (ii) use the trained algorithm to process data indicative of a presence, absence, or relative assessment of urologic condition (e.g., bladder cancer, kidney cancer, and prostate cancer) of a subject, (iii) determine a quantitative measure indicative of a presence, absence, or relative assessment of urologic condition of a subject, (iv) identify or provide an indication of the urologic condition of the subject, or (v) electronically output a report that identifies or provides an indication of the urologic condition of the subject.

### EXAMPLES

### Example 1—Optimization of Sequencing Library Methods

A hybrid capture library preparation method is designed and optimized to perform cost-effective sensitive detection of low-abundance bladder cancer mutations present in urine-derived DNA.

A custom hybrid capture probe set is designed, and a set of oligos is manufactured, for a set of bladder cancer-associated genes encompassing over 140,000 bases. A set of 1,500 oligonucleotide sequences is optimized in silico to avoid off-target enrichment and to promote uniform binding thermodynamics. Custom laboratory methods are optimized utilizing sequential capture reactions, the DNA input concentration into the hybrid capture reaction is optimized, and a number of PCR amplification cycles both pre-capture and post-capture are established.

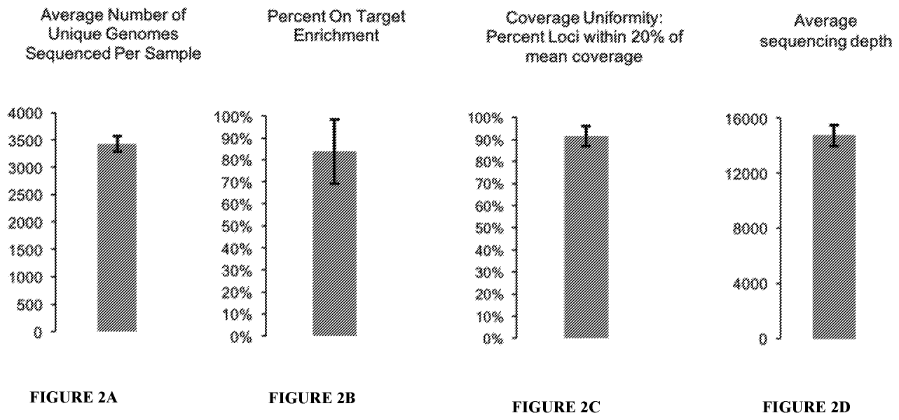

These optimizations increase on-target efficiency and maximize coverage uniformity and sequencing depth in targeted sequencing libraries (as shown in FIG. 2). FIG. 2 illustrates development of a custom hybrid capture panel to analyze 140,000 bladder cancer disease loci. (A) Average number of unique genomes analyzed per sample. This level of library complexity allows a bladder cancer detection algorithm to implement multiple methods of noise suppression while confidently calling mutations at frequencies of as low as 1:1000 genomes. (B) Percent on target enrichment. Of all sequencing reads analyzed, over 80% are dedicated to our genes/loci of interest, and the remaining 20% “off-target” loci allow copy number variation algorithms to normalize hybrid capture performance within a sample. Compared to published technical performance of standard hybrid capture methods, this double capture approach achieves 30-40% higher on-target efficiency, achieving equivalent reductions in the amount of sequencing required (cost reduction). (C) Uniformity of coverage is achieved where greater than 96% of loci achieve coverage depth within 20% of the mean coverage. (D) Average sequencing depth. This high level of uniform coverage results in fewer low coverage loci and maximizes sensitivity across the panel. All values are average of 29 reference samples, error bars denote standard error of the mean. Further, the economical and targeted use of the UriSeq reagents enables a scalable sequencing library approach that provides commercially viable and cost-effective up to about 15,000× depth sequencing.

Samples are typically run at 100, 500, 1000, or 5000× X depth, and there are 140,000 base pairs per sample, thereby yielding 2.1 billion bases analyzed per sample. Samples are run using a HiSeq 2500 with a capacity of 600 million paired end reads×250 bp read length=150 billion bases of capacity per sequencing run. Each reaction can process a total of 150 billion/2.1 billion=71 samples multiplexed per run.

### Example 2—Evaluation of Standard Mutation Callers in Healthy Controls and Bladder Cancer Patients

The sensitivity and specificity of the Broad Institute's MuTect Algorithm, a best-in class mutation algorithm used for solid tumors, is benchmarked and evaluated. A set of 15 healthy controls and a cohort of 6 patients with verified high-grade bladder cancer are investigated. Urine samples are collected from patients with cancer prior to surgical removal of their tumor. Among the bladder cancer cohort, the genomic signatures of bladder cancer in peripheral blood (negative control), flash frozen tumor (positive control), and urine voids (experimental test case) are analyzed. The MuTect algorithm is applied to tumor sequencing data to define true positive mutational events. With this cancer baseline established, MuTect is then used to evaluate mutational signatures in urine-derived DNA. The percentage of true positives detected in the urine is quantified to establish the concordance of tumor variants and urine variants detected by MuTect.

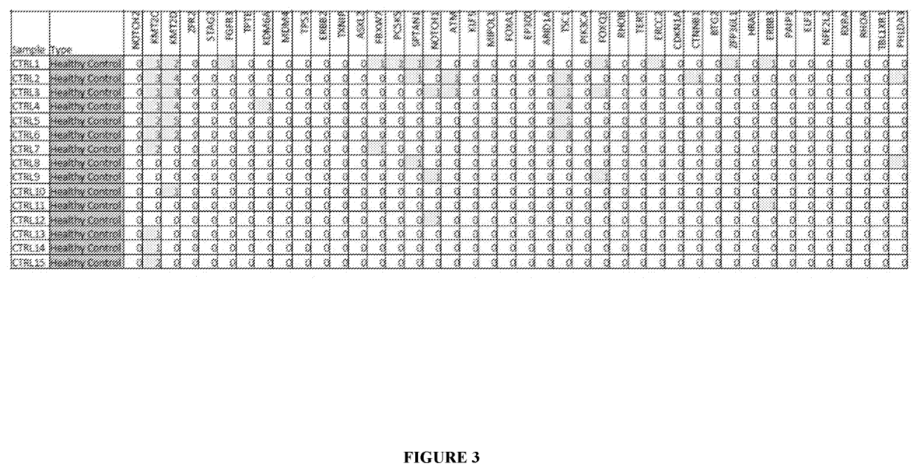

When using the MuTect algorithm on healthy control subjects, all control subjects are found to have one or more false positive mutation calls (as shown in FIG. 3), suggesting a substantial limitation in specificity. FIG. 3 illustrates results showing that best-in-class mutation callers typically report a substantial number of false-positive mutations in deep sequencing of urine. Results from the Broad Institute's MuTect algorithm are reported for high-depth DNA sequencing of urine samples obtained from 15 healthy control subjects. Each column represents a gene, and each row represents a control urine sample. Young healthy controls are selected that have no history of cancer and with urine chemistries within normal range (no abnormalities in the 10 urine analytes measured). Selection of healthy normal urine is used to minimize the likelihood of true mutations and instead to illustrate the degree of false-positive mutation calls due to use of a mutation calling algorithm not optimized for the types of technical noise present in urine sequencing data. Each shaded box denotes a mutation called by MuTect in a gene (columns) and patient (rows), numbers in the boxes denote the number of events called within a gene where approximately half of positive samples have multiple false-positive mutations called within an individual gene. All control subjects are found to have one or more false-positive mutation calls. This data serves as a significant rationale for development of an improved diagnostic-grade mutation caller.

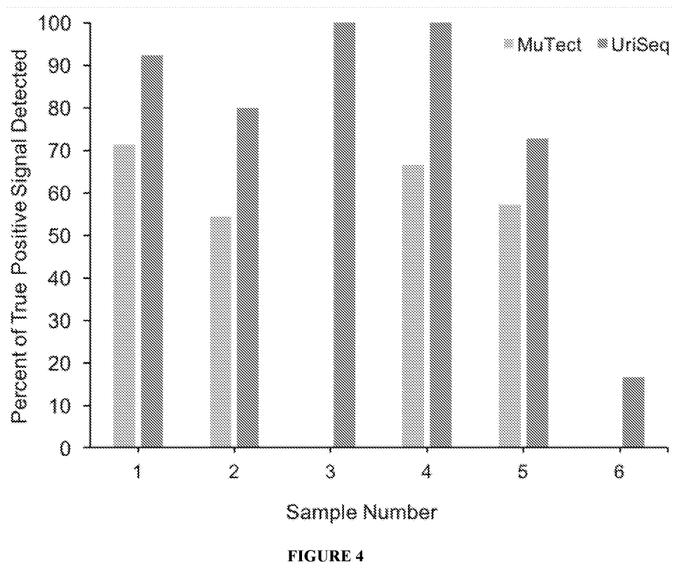

In cancer samples, MuTect is found to be insufficient for clinical use in bladder cancer, as it detected only 41% of true-positive tumor events in urine. Significantly, this limited sensitivity is most pronounced in about ⅓ of cancer samples in the study, where no mutational events in the urine samples were detected by MuTect (as shown in FIG. 4). FIG. 4 illustrates results showing superior detection of tumor true positives in urine by UriSeq. The UriSeq and MuTect algorithms are used to define true-positive events in tumor DNA. The same algorithm is then used to detect the same mutational events in urine-derived DNA. The percentage of true positives detected quantifies the concordance of tumor variants and urine variants detected by the same algorithm. On average, UriSeq detected 77% of known true positives compared to only 41% by MuTect. UriSeq detected tumor signal in 100% of samples tested while MuTect failed to detect tumor signal in urine in 33% of samples. These two samples where MuTect failed on sensitivity were defined by lower allele frequency events. MuTect has been validated to call variants above 5% allele frequency.

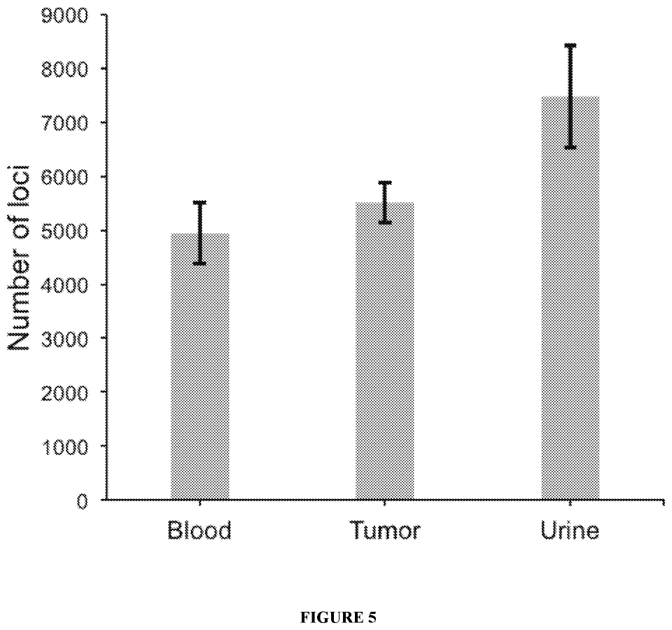

To better understand MuTect's technical limitations, the level of noise in urine raw sequencing data was analyzed, and 34% more noise (non-reference matching loci) was found in urine-derived DNA compared to blood-derived DNA from the same individual, and 26% more noise was found in in urine-derived DNA than frozen tumor-derived DNA from the same individual (as shown in FIG. 5). FIG. 5 illustrates results showing that non-reference events are more prevalent in urine sequencing. In a 6-patient study, technical sequencing noise is investigated in paired peripheral blood, tumor, and urine samples collected from patients just prior to surgical removal of the tumor. The noise profile (defined as the number of non-reference events with alternate allele frequencies in our target detection range of 0.15% to 30%) is quantified. The mean number of loci contributing to noise across sample type is reported. Error bars denote standard error of the mean. These data demonstrate 34% more noise in urine-derived DNA compared to blood-derived DNA from the same individual, and 26% more noise in urine-derived DNA compared to tumor-derived DNA from the same individual.

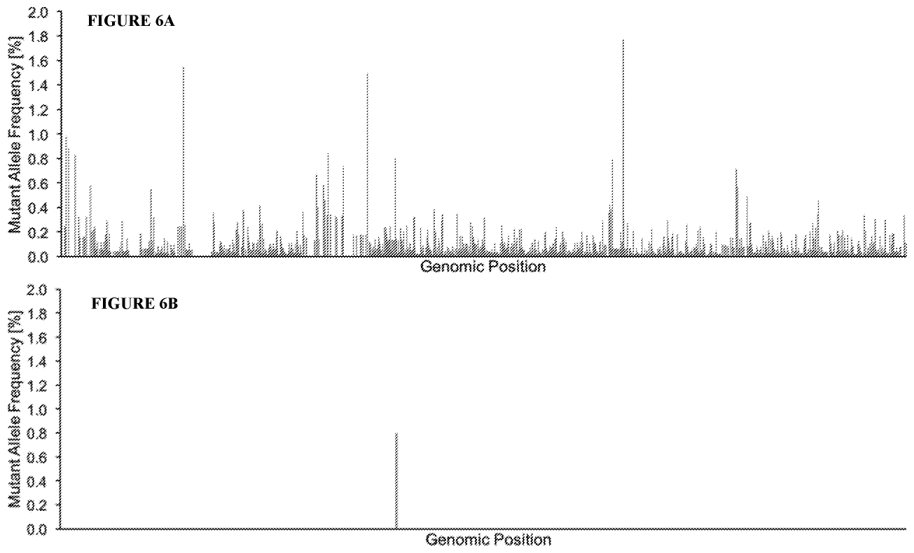

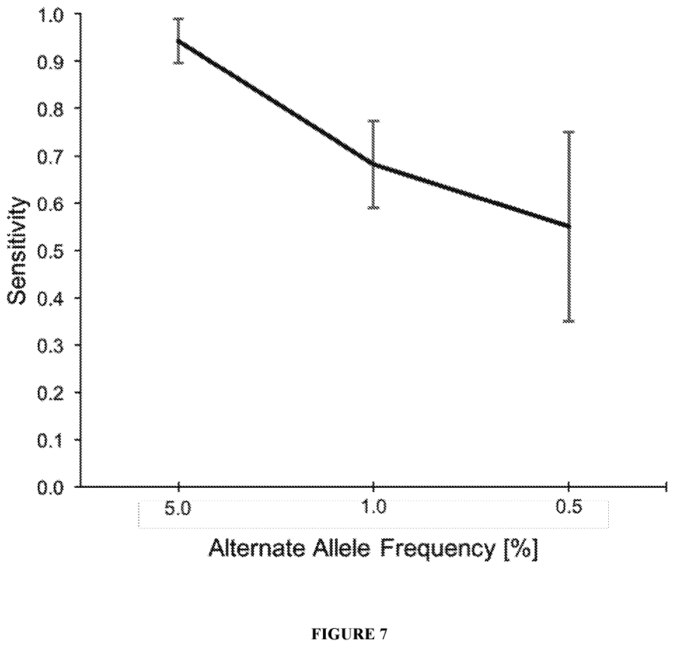

FIG. 6 illustrates results showing that UriSeq noise suppression distinguishes noise from confident true-positive low-frequency mutations. (A) Representative putative mutational profile (non-reference signal present in raw data) of urine-derived DNA and (B) UriSeq algorithmic filtering (removal of noise signal) and identification of a high confidence mutational event (orange). These data are derived from a patient with bladder cancer and generated via analysis of matched tumor and urine samples. The vertical axis represents non-reference allele frequency, and the horizontal axis denotes genomic base pair location within the gene KDM6A. The detected signal in urine (B), orange bar, is confirmed by a shared mutation signal found in sequencing the pure matched tumor. This mutation call is further supported as it was previously identified by The Cancer Genome Atlas (TCGA) Project and cBio Database as a hotspot loss of function mutation in other patients with bladder cancer. In this patient, the tumor signal is diluted by normal contaminating DNA in the urine such that the tumor signal intensity falls into the range of typical sequencing noise (A). UriSeq's error suppression approach that (i) utilizes paired-end sequencing to correct sequencing errors, (ii) performs labeling and tracking of unique sequencing molecules within PCR duplicate copies to suppress both PCR and sequencing induced errors, (iii) utilizes the duplex nature of double-stranded DNA by defining read family strand information to quantify and mitigate DNA damage artifacts, and (iv) performs empirical modeling of noise profiles at each of the 140,000 loci using 25 reference urine DNA samples to remove all false-positive signal, leaving only the one true positive tumor matched event within KDM6A.

### Example 3—Variant Detection Algorithm Development Using Blinded Testing on Cancer and Normal Samples

Approaches to detect bladder cancer in urine samples, such as MuTect, may perform with inadequate sensitivity and specificity to support a clinical grade diagnostic product in urine. Further, approaches such as MuTect may require a paired sample cancer-blood analysis which adds prohibitive cost and logistic complexity in the clinic. These challenges demonstrate an urgent need for a tailored urine-based clinical grade mutation caller for bladder cancer. Therefore, a bladder cancer disease classification algorithm (e.g., UriSeq) is developed in a training setting, in which the disease status of each sample is known a priori.

To enhance genomic variant sensitivity and specificity in the presence of urine-induced DNA damage, a collection of 80 metrics is developed and computed for each of the 140,000 loci in a targeted gene panel, to circumvent both platform-derived errors (e.g., sequencing and PCR errors) and urine-induced DNA damage errors. These metrics enable the development of a mutation detection algorithm that quantitatively mitigates sources of ambiguity in urine-derived DNA by leveraging four methods of error suppression: (i) paired-end sequencing to correct sequencing errors; (ii) labeling and tracking of unique sequencing molecules within PCR duplicate copies to suppress both PCR and sequencing induced errors; (iii) utilizing the duplex nature of double stranded DNA to examine concordance of mutation calls on sense and antisense strands of an original molecule and thereby mitigate DNA damage artifacts; (iv) empirical modeling of noise profiles at each of the 140,000 loci using 18 reference urine DNA samples; and (v) assessing the location of the putative single nucleotide variants position relative to: the sequencing read cycle/location within the sequencing read and/or the location of the putative single nucleotide variant within the total nucleic acid fragment, in particular its proximity to the nucleic acid molecules ends.

To develop appropriate thresholds for these metrics to identify bladder cancer mutations and establish an experimental limit of detection within the UriSeq assay, two reference samples are diluted with predetermined SNP loci into each other at 1:10, 1:50, 1:100 in duplicate; and this design is repeated with four independent reference samples. Next, the performance of the algorithm is validated for identifying relevant disease associated variants in patients with known bladder cancer, by applying the algorithm to both tumor and urine samples in a series of 6 paired samples comprising blood, tumor, and urine specimens (e.g., as described in Example 2). Next, the percentage of true positives detected in the urine is quantified to establish the concordance of tumor variants and urine variants detected by UriSeq. Finally, using diverse bladder cancer and non-bladder cancer sample cohorts, a set of 50 control and 50 bladder cancer samples was randomly selected to validate the performance of the mutation calling algorithm (as shown in Table 2 and Table 3).

TABLE 2
Summary of UriSeq clinical study cohorts

n

Bladder Cancer Clinical
Demographics
Gender

| | |
|---|---|
| Female | 10 |
| Male | 40 |

Tumor Stage

| | |
|---|---|
| Ta | 23 |
| T1 | 6 |
| T2 | 10 |
| T3 | 8 |
| T4 | 2 |
| Tx | 1 |

Tumor Grade

| | |
|---|---|
| Low | 11 |
| Medium | 5 |
| High | 33 |
| Gx | 1 |

Surgery Type

| | |
|---|---|
| Transurethral Resection | 32 |
| Radical Cystectomy | 17 |
| Nephroureterectomy | 1 |

Non-Bladder Cancer Clinical
Demographics
Gender

| | |
|---|---|
| Female | 19 |
| Male | 31 |

Active Cancers

| | |
|---|---|
| Prostate | 2 |

Past Cancers

| | |
|---|---|
| Prostate | 4 |
| Melanoma | 3 |
| Renal Cell Carcinoma | 2 |
| Basal Cell Carcinoma | 2 |
| Small Cell Lung Cancer | 1 |
| Uterine | 1 |
| Pancreatic | 1 |
| Esophagheal | 1 |
| SCC of the throat | 1 |

Urologic Conditions

| | |
|---|---|
| Benign Prostate Hyperplasia | 13 |
| Hematuria | 9 |
| Lower Urinary Tract Symptoms | 8 |
| Kidney Stones | 2 |
| Prostatitis | 1 |

Table 2 shows the clinical characteristics of 50 patients with bladder cancer (left) and 50 non-cancer controls (right) used to establish the clinical performance of UriSeq.

TABLE 3
Summary of UriSeq Clinical Diagnostic
Performance on a Validation Cohort

| | Number of | |
|---|---|---|
| Disease Classification | Samples | Clinical Features, Notes |
| True Positives | 45 | |
| True Negatives | 49 | |
| False Positives | 1 | Previous prostate cancer |
| False Negatives | 5 | Small tumors, low grade disease, |
| | | and 1 sample borderline on |
| | | sample exclusion QC metric |
| | | threshold |

As shown in Table 3, in a test on a set of randomly selected 50 non-cancer control and 50 bladder cancer samples, observed classifications included 45 true positives, 49 true negatives, 1 false positive, and 5 false negatives; thereby yielding a clinical sensitivity of 90%, clinical specificity of 98%, positive predictive value (PPV) of 98%, and negative predictive value (NPV) of 91%. Of note, the 1 false positive case is an 85-year-old patient being monitored after prostate cancer treatment. The false negative cases are enriched for low grade disease, one patient with a very small tumor, and one sample that is borderline on sample quality control performance metrics. Further validation studies can be performed with larger sample cohorts to further refine sample QC requirements and to adjust disease classification rules, thereby enhancing classification performance of the algorithm.

Using the dilution experiments of urine reference samples, the trained algorithm is trained using metrics from the 4 error suppression metrics described above, thereby developing empirical cutoffs. To account for urine-specific sequencing noise in the algorithm, technical specificity is initially prioritized to minimize future false positive disease classification. Of 125 billion bases analyzed in the dilutions, the algorithms' stringency is optimized so that no false positives are called. Following training of the algorithm for maximal specificity, at a 5% variant allele frequency (1:10 dilution of heterozygous loci), an average of 95% of variants are detected. At a 1% variant allele frequency (1:50), an average of 70% of variants are detected. At a 0.5% variant allele frequency (1:100), an average of 55% of variants are detected (as shown in FIG. 7).

FIG. 7 illustrates results showing UriSeq variant detection algorithm sensitivity at various dilution levels. In response to the performance challenges observed in the MuTect algorithm, a urine-derived DNA optimized mutation caller is developed with extremely high specificity. In this experiment, 27 serial dilution samples are sequenced at high depth using known reference samples, and an algorithm is developed where stringency is set to eliminate all false positive calls. Among these 27 samples, 250 billion bases were analyzed with 0 false positive calls, establishing a specificity of less than 1 false positive per 250 billion bases analyzed. With this specificity, the presented sensitivity is achieved such that at a dilution where true-positive variants are present at 5% frequency, more than 94% of variants are correctly identified. When diluted to 1% frequency, more than 68% of variants are correctly identified. When diluted to 0.5%, more than 55% of variants are correctly identified.

With optimized metric thresholds and incorporation of this noise model in a study across 6 tumor-urine pairs, a total of 68 mutations are identified in tumor and 56 mutations are identified in urine. Of mutations identified in tumor by UriSeq, 77% are also found in urine, compared to only 41% using MuTect (as shown in FIG. 4). UriSeq correctly classifies 100% of urine cancer samples while MuTect fails to detect tumor signal in urine in 33% of samples.

Overall, the training performance of the algorithm is found to be sufficient. Further, the classification is performed with clinical grade sensitivity and specificity. The UriSeq assay overcomes multiple challenges in urine-derived DNA sequencing that may limited low-frequency variant or mutation measurements to single nucleotide genotyping at a set of known hotspot loci. Through implementation of multi-pronged noise suppression methods, combined with tailored molecular biology, excellent assay performance (e.g., clinical specificity and sensitivity) is demonstrated in tumor mutation callers to permit disease diagnosis and monitoring tumor recurrence or evolution from urine-derived DNA. The optimization of both molecular biology and algorithmic components of UriSeq enable reduced assay costs, allowing commercial viability in multiple medical diagnostic indications. The urine mutation calling approach has further potential utility in the diagnosis and characterization of many disease states of the urologic system. These methods can be applied to other biologic indications, such as predicting therapeutic response to targeted cancer agents, diagnosis of prostate and kidney cancers, and basic research explorations of low-frequency mutagenesis and development of clonal stem cell populations in response to carcinogen exposures. These foundational bioinformatics methods can support guided development of urine preservation buffers and DNA extraction methods to enable new clinical approaches for a host of diseases that can be monitored via urine.

### Example 4—Sequencing Metrics to Quantitatively Mitigate Sources of Allele Measurement Error in Urine-Derived DNA

Sequencing approach described herein can leverage three methods of error suppression: (i) paired-end sequencing to correct sequencing errors, (ii) labeling and tracking of unique sequencing molecules within PCR duplicate copies to suppress both PCR and sequencing induced errors, and (iii) utilizing the duplex nature of double stranded DNA to examine concordance of mutation calls on sense and antisense strands of an original molecule and thereby mitigate DNA damage artifacts. Companion metrics to this prescribed sequencing approach can be used to enable quantitative mitigation of sources of allele measurement error in urine-derived DNA.

To account for DNA damage, PCR errors, and sequencing errors in sample data, a host of companion metrics to sequencing approaches described herein are defined and computed at each base location (genomic position) in a set of genomic regions of interest. These metrics enable DNA measurement quality control, quality assurance, and provide a means to conduct high confidence single nucleotide variant (SNV) detection. For example, these metrics (shown in Table 4) can be used to establish quality control of samples and enable high-confidence detection of single nucleotide variants associated with cancer and non-pathologic single nucleotide polymorphisms

TABLE 4
Sequencing metrics to quantitatively mitigate sources
of allele measurement error in urine-derived DNA

| Column Name | Description |
|---|---|
| sampleID | Patient or Dilution Independent Identifier |
| gene | HUGO Identifier |
| chrom | Chromosome |
| base_loc | Base pair coordinate |
| ref_allele | Reference allele at this base pair location |
| alt_allele | Second most prevalent allele at this base pair location |
| VCF mutation | Haplotype Caller Annotation: Denotes if this |
| | base pair was detected as a true positive |
| DBSNP | Annotates if base position is in the dbsnp |
| | reference of prevalent/nonpathogenic mutations |
| TCGA | To be completed: Annotates if base position |
| | is in the TCGA mutation database for any cancer |
| total_reads | Total Reads (including duplicates) that |
| | cover the position of interest |
| total_reads_phred_fil t | Total reads at a location |
| nonref_a | Total reads at a location - does not include |
| | duplicates and only includes reads with |
| | phred >=30 |
| nonref_a_phred | percentage of reads at a location that are |
| | non-reference “a” reads |
| nonref_a_mapq | associated mean phred score of non-reference |
| | “a” reads |
| nonref_c | associated mean mapping quality score of |
| | non-reference “a” reads |
| nonref_c_phred | percentage of reads at a location that |
| | are non-reference “c” reads |
| nonref_c_mapq | associated mean phred score of non-reference |
| | “c” reads |
| nonref_g | associated mean mapping quality score of |
| | non-reference “c” reads |
| nonref_g_phred | percentage of reads at a location that |
| | are non-reference “g” reads |
| nonref_g_mapq | associated mean phred score of non-reference |
| | “g” reads |
| nonref_t | associated mean mapping quality score of |
| | non-reference “g” reads |
| nonref_t_phred | percentage of reads at a location that are |
| | non-reference “t” reads |
| nonref_t_mapq | associated mean phred score of non-reference |
| | “t” reads |
| ref_a | associated mean mapping quality score of |
| | non-reference “t” reads |
| ref_a_phred | percentage of reads at a location that are |
| | reference-matching “a” reads |
| ref_a_mapq | associated mean phred score of reference-matching |
| | “a” reads |
| ref_c | associated mean mapping quality score of |
| | reference-matching “a” reads |
| ref_c_phred | percentage of reads at a location that are |
| | reference-matching “c” reads |
| ref_c_mapq | associated mean phred score of reference-matching |
| | “c” reads |
| ref_g | associated mean mapping quality score of reference- |
| | matching “c” reads |
| ref_g_phred | percentage of reads at a location that are reference- |
| | matching “g” reads |
| ref_g_mapq | associated mean phred score of reference-matching |
| | “g” reads |
| ref_t | associated mean mapping quality score of reference- |
| | matching “g” reads |
| ref_t_phred | percentage of reads at a location that are reference- |
| | matching “t” reads |
| ref_t_mapq | associated mean phred score of reference-matching |
| | “t” reads |
| total_num_fam | associated mean mapping quality score of reference- |
| | matching “t” reads |
| avg_fam_size | Average number of PCR duplicated-reads that make up |
| | each family |
| max_fam_size | The number of PCR duplicated reads in the largest family |
| | covering the position of interest |
| total_ref_reads | Total number of reads that match the reference allele |
| total_alt_reads | Total number of reads that match the alternate allele |
| total_collision_reads | Total number of reads that represent a collision |
| total_error_reads | Total number of reads that represent an error: error defined |
| | as reads that do not match the reference, nor alternate. |
| error_rate | Error reads/Total number of reads |
| collision_rate | Collision reads/Total number of reads |
| alt_allele_freq | Frequency of the second-most prevalent allele at the |
| | position of interest |
| total_num_families_filtered | Total number of duplicate families that contain at least 1 |
| | alternate allele. |
| avg_fam_size_filtered | Average number of PCR duplicated-reads that make up |
| | each family* |
| purity_measure_colli sions | 100 − (The number of families in which a collision |
| | occurs/total number of families) |
| purity_measure_pure fams | Number of families that are purely the mutant allele/Total |
| | number of families |
| max_fam_size_filtere d | The number of PCR duplicated reads in the largest family |
| | covering the position of interest. |
| total_reads_filtered | Total reads covering position of interest* |
| total_ref_reads_filtered | Total number of reads that match the reference allele* |
| total_alt_reads_filtered | Total number of reads that match the alternate allele* |
| total_collision_reads_filtered | Total number of reads that represent a collision* |
| avg_fam_size_pure_families | Average number of PCR duplicated-reads that make up |
| | each family (Family is defined by having at least one |
| | alternate allele) |
| Collision | A collision occurs when reads from the same PCR-derived |
| | family have more than one allele represented in the |
| | sequencing read at the position of interest. |

### Example 5—a Hybrid Capture Panel Design Strategy for Urologic Specificity

A hybrid capture panel design strategy can be developed to achieve urologic specificity in detection of and/or distinguishing between different diseases, disorders, or conditions, such as urologic cancers (e.g., bladder cancer, kidney cancer, and/or prostate cancer). Using methods and systems of the present disclosure, biological samples can be analyzed at specific panels of genes to determine tissue type, organ or cell type of origin. For example, the top 5 genes that are differentially measured among cancer vs. healthy patients can be identified for each of a plurality of different urologic cancers (e.g., cancer of different tissues including bladder cancer, kidney cancer, and/or prostate cancer). For example, the 5 genes that are differentially measured among kidney cancer vs. healthy patients are VHL, PBRM1, MUC, TTN, and SETD1, with 45%, 29%, 15%, 13%, and 11% of a plurality of kidney cancer patients having observable mutations in the gene, respectively. As another example, the 5 genes that are differentially measured among prostate cancer vs. healthy patients are ERG, TP53, MUC16, SPOP, and SYNE1, with 30%, 18%, 11%, 9%, and 7% of a plurality of prostate cancer patients having observable mutations in the gene, respectively. As another example, the 5 genes that are differentially measured among bladder cancer vs. healthy patients are TP53, KDM6A, MLL2, ARID1A, and PIK3CA, with 50%, 29%, 28%, 25%, and 22% of a plurality of bladder cancer patients having observable mutations in the gene, respectively.

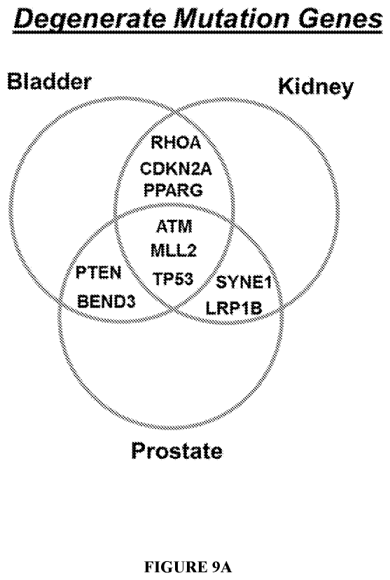

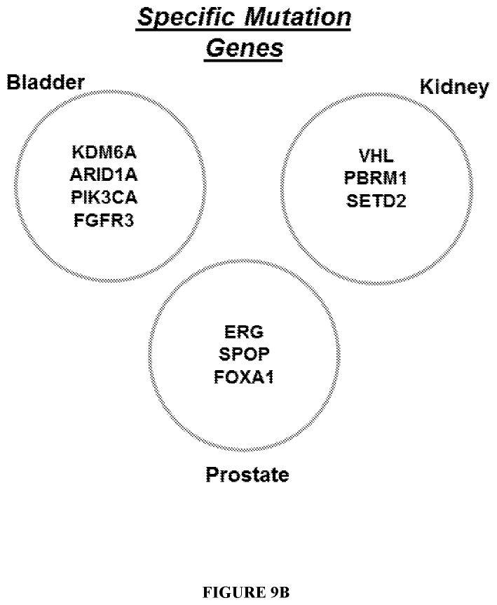

FIGS. 9A and 9B illustrate a hybrid capture panel design strategy for urologic specificity, based on selection of gene panels for detection of bladder cancer, kidney cancer, and prostate cancer, which may comprise degenerate mutation genes and/or specific mutation genes, respectively. Degenerate mutation genes may be genes having observed mutations in multiple types of urologic cancers (e.g., two of more of: bladder cancer, kidney cancer, and prostate cancer). For example, a panel of degenerate mutation genes for bladder cancer may include RHOA, CDKN2A, PPARG, ATM, MLL2, TP53, PTEN, and BEND3. As another example, a panel of degenerate mutation genes for kidney cancer may include RHOA, CDKN2A, PPARG, ATM, MLL2, TP53, SYNE1, and LRP1B. As another example, a panel of degenerate mutation genes for prostate cancer may include PTEN, BEND3, ATM, MLL2, TP53, SYNE1, and LRP1B. Alternatively or in combination, panels of specific mutation genes may be chosen, which are genes specific for a particular urologic cancer among the plurality of urologic cancers (e.g., only one of: bladder cancer, kidney cancer, and prostate cancer). For example, a panel of specific mutation genes for bladder cancer may include KDM6A, ARID1A, PIK3CA, and FGFR3. As another example, a panel of specific mutation genes for kidney cancer may include VHL, PBRM1, and SETD2. As another example, a panel of specific mutation genes for prostate cancer may include ERG, SPOP, and FOXA1.

A hybrid capture panel design strategy for urologic specificity may also comprise complementary measurements of selected gene panels comprising genes having copy number variation (CNV) for complex biology cases. As shown in Table 5, genes observed to have CNV in some complex biology cases include ARID1A, ASXL2, ATM, ERBB3, ERCC2, MLL2, NOTCH2, PIK3CA, RHOA, TP53, and TPTE. For example, such genes may be observed to have either CNV gain or CNV loss. Further, different genes may be enriched in low-grade (LG) vs. high-grade (HG) disease.

TABLE 5
Complementary measurements of genes having CNV for complex biology cases

| | | | | mut_gene | mut_gene | mut_gene | CNV | CNV |
|---|---|---|---|---|---|---|---|---|
| Sample | Stage | Grade | AAF (%) | 001 | 002 | 003 | gain | loss |
| 1 | T4 | HG | 4.14 | TP53 | NOTCH2 | | 3 | |
| 2 | Ta | LG | 4.24 | TP53 | FGFR3 | | 1 | 3 |
| 3 | Ta | HG | 2.59 | ASXL3 | FGFR3 | | | 4 |
| 4 | Ta | LG | 0.63 | NOTCH2 | ATM | | 1 | 2 |
| 5 | Ta | LG | 1.08 | FGFR3 | KDM6A | | | 1 |
| 6 | T2 | HG | 3.81 | ARID1A | TP53 | TP53 | | 4 |
| 7 | T3 | HG | 7.94 | MLL2 | TPTE | RHOA | 3 | 1 |
| 8 | T2 | HG | 14.32 | ERBB3 | TP53 | ARID1A | 1 | 14 |
| 9 | T2 | HG | 58.96 | TP53 | ERCC2 | PIK3CA | 25 | 9 |

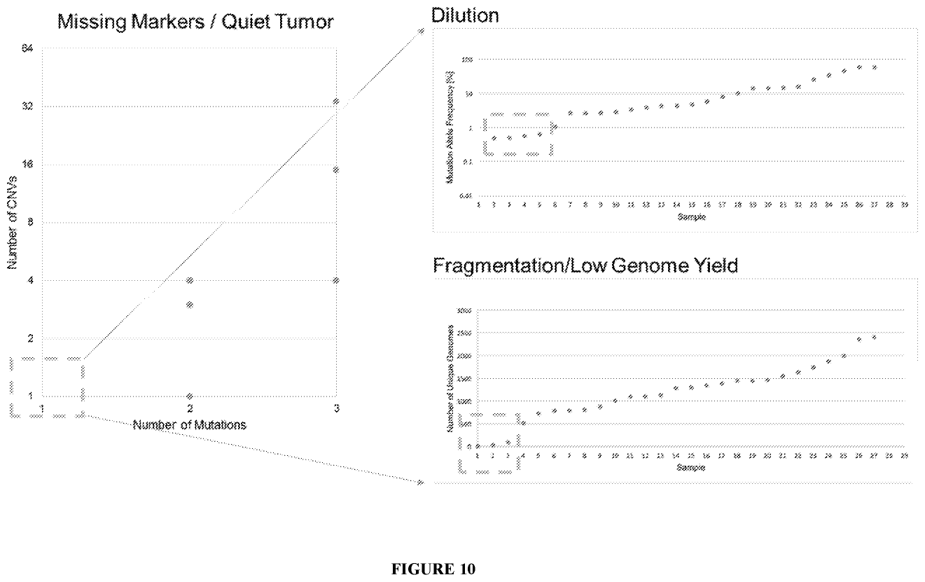

A hybrid capture panel design strategy for urologic specificity may also comprise measurements of selected gene panels of informative genes or loci having dynamic behaviors of DNA fragmentation and read depth coverage profiles specific to a tissue type or cell type of origin. For example, FIG. 10 illustrates a “missing markers/quiet tumor” case in which both the number of CNVs and the number of mutations are low (left). Some samples have low mutation allele frequency (%) due to dilution (top right), and some samples have a low number of unique genomes due to fragmentation and low genome yield (bottom right).

### Example 6—Model Training and Validation for Bladder Cancer

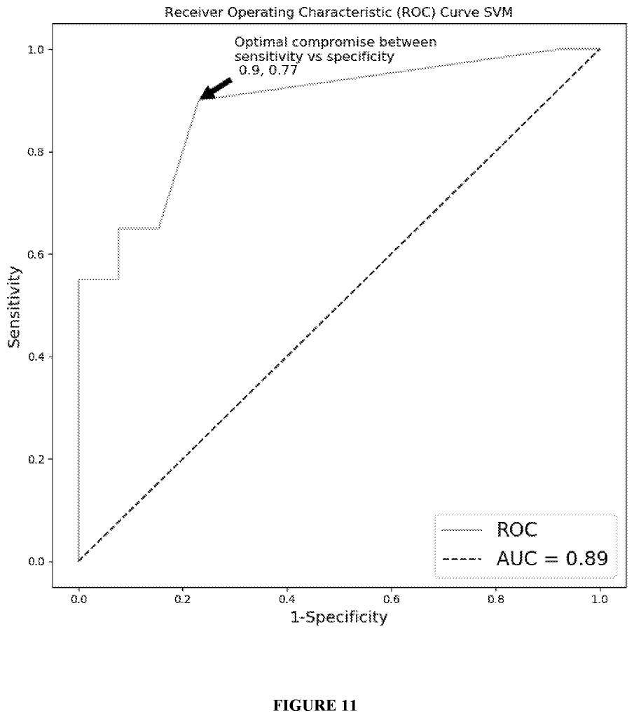

As an illustrative example, bladder cancer patient samples were assessed using a grade prediction model and machine training and validation. It is understood that this model is applicable for other cancers, and specifically urologic cancers and conditions as described herein. As illustrated in FIG. 11 shows a graph illustrating a Model Training: Receiver Operating characteristic (ROC) curve for Bladder Cancer grade prediction. ROC was performed for calibrated Support Vector Machine (SVM) classifier using 10-fold cross validation. The problem was structured as a binary supervised learning with high grade tumor as positive label. In the ROC curve, the true positive rate (sensitivity) is plotted as a function of the false positive rate (1−specificity). The total number of subjects is 553 which 489 labeled high grade and 64 low grades. The area under the curve, AUC, is 0.89 which indicates the power of separability of the trained model.

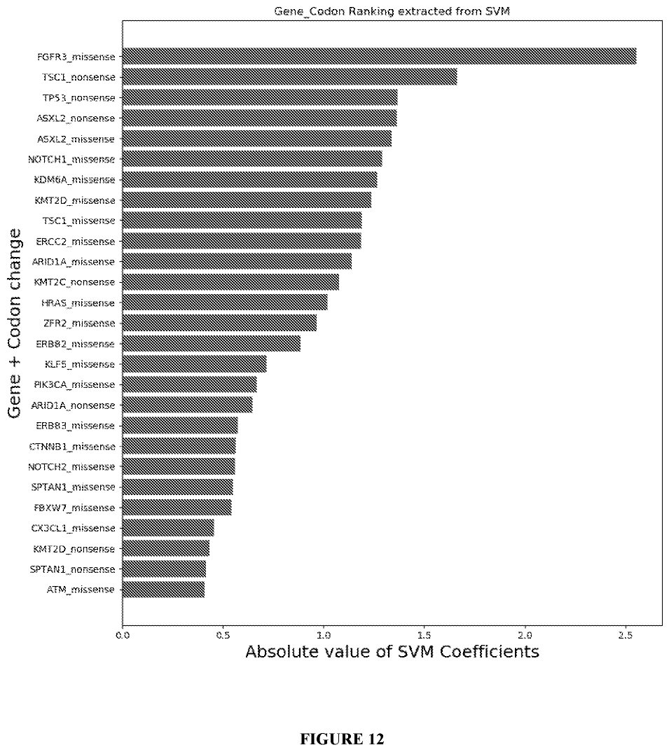

FIG. 12 shows properties of the trained model: ranking the genes in prediction of BLCA grade. The final data consists of 553 subjects and 75 risk factors. The risk factors were engineered by combining mutated gene and amino acid changes either to missense or nonsense. 75 risk factors are ranked based on their contribution in the predictive power of the final classifier positively or negatively.

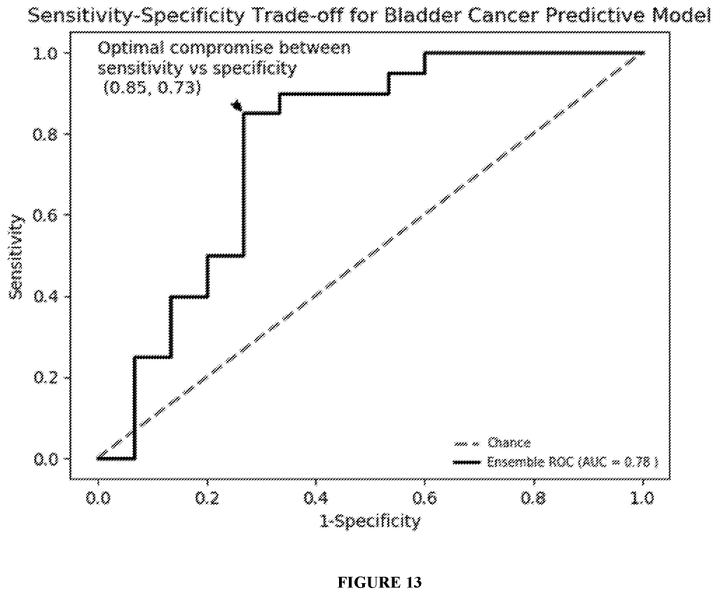

FIG. 13 shows a graph illustrating the Model Validation: Receiver Operating Characteristic (ROC) curve for Bladder Cancer grade prediction. After training the model (an ensemble support vector machine classifier) we explored the validity of the model on a cohort comprised of 35 individuals (LG=15, HG=20) whose urine-based DNA sequencing was inputted into the model. Grade was predicted with a sensitivity of 85% and specificity of 73%.

Accordingly, this Example shows that by machine learning and training the model, the grade and origin of nucleic acid in the sample can be determined with a high degree of sensitivity and specificity.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for identifying or monitoring a urologic condition of a subject comprising:
(a) processing a biological sample obtained or derived from said subject to generate a dataset, wherein said dataset is indicative of a presence, absence, or relative assessment of said urologic condition of said subject;
(b) using a trained algorithm to process said dataset to determine a quantitative measure indicative of said presence, absence, or relative assessment of said urologic condition of said subject;
(c) based at least in part on said quantitative measure, identifying or providing an indication of said urologic condition of said subject with one or more of: (i) a sensitivity of at least about 90%, (ii) a specificity of at least about 90%, (iii) a positive predictive value of at least about 90%, and (iv) a negative predictive value of at least about 90%; and
(d) electronically outputting a report that identifies or provides an indication of said urologic condition of said subject.

2. The method of claim 1, wherein said biological sample is urine or a derivative thereof.

3. (canceled)

4. The method of claim 1, wherein processing said biological sample comprises polymerase chain reaction (PCR).

5. The method of claim 1, wherein (c) comprises identifying or providing an indication of said urologic condition of said subject with two or more of: (i) a sensitivity of at least about 90%, (ii) a specificity of at least about 90%, (iii) a positive predictive value of at least about 90%, and (iv) a negative predictive value of at least about 90%.

6-7. (canceled)

8. The method of claim 1, wherein (c) comprises identifying or providing an indication of said urologic condition of said subject with a sensitivity of at least about 90%.

9-13. (canceled)

14. The method of claim 1, wherein (c) comprises identifying or providing an indication of said urologic condition of said subject with a positive predictive value (PPV) of at least about 90%.

15-19. (canceled)

20. The method of claim 1, wherein (c) comprises identifying or providing an indication of said urologic condition of said subject with an Area Under Curve (AUC) of at least about 0.90.

21-22. (canceled)

23. The method of claim 1, wherein (a) comprises (i) subjecting said biological sample to conditions that are sufficient to isolate, enrich, or extract a plurality of nucleic acid molecules, and (ii) assaying said plurality of nucleic acid molecules to generate said dataset.

24. The method of claim 23, further comprising extracting a plurality of DNA molecules from said biological sample, and subjecting said plurality of DNA molecules to sequencing to generate a plurality of sequencing reads, wherein said dataset comprises said plurality of sequencing reads.

25. The method of claim 24, wherein said sequencing is massively parallel sequencing.

26. The method of claim 24, wherein said sequencing is performed at a depth of at least about 100× to 5,000×.

27. The method of claim 26, wherein said sequencing is performed at a depth of at least about 100-1000×.

28. (canceled)

29. The method of claim 24, wherein said sequencing comprises nucleic acid amplification.

30. The method of claim 29, wherein said nucleic acid amplification comprises polymerase chain reaction (PCR).

31. (canceled)

32. The method of claim 24, further comprising using probes configured to selectively enrich said plurality of nucleic acid molecules corresponding to a panel of one or more genomic loci.

33-34. (canceled)

35. The method of claim 32, wherein said panel of said one or more genomic loci comprises at least 50,000 distinct genomic loci.

36. (canceled)

37. The method of claim 24, further comprising performing error suppression of said plurality of sequence reads by one or more of: (i) paired-end sequencing to correct sequencing errors, (ii) labeling and tracking of unique sequencing molecules within amplicons to suppress PCR and sequencing-induced errors, (iii) examining concordance of mutation calls on sense and antisense strands of said plurality of DNA molecules, (iv) suppression of noise profiles at said panel of one or more genomic loci using a plurality of reference cell associated and/or cell-free biological DNA samples, or (v) assessing the location of a putative single nucleotide variant position relative to the sequencing read cycle or location within the sequencing read and/or the location of the putative single nucleotide variant within the nucleic acid fragment and its proximity to the end of the fragment.

38-40. (canceled)

41. The method of claim 1, wherein said biological sample is processed without nucleic acid isolation, enrichment, or extraction.

42. The method of claim 1, wherein said report is presented on a graphical user interface of an electronic device of a user.

43-49. (canceled)

50. The method of claim 1, further comprising providing said subject with a therapeutic intervention for said urologic condition.

51. The method of claim 50, wherein said therapeutic intervention comprises surgery, chemotherapy, radiotherapy, immunotherapy, or a combination thereof.

52. The method of claim 1, further comprising monitoring said urologic condition, wherein said monitoring comprises assessing said urologic condition of said subject at a plurality of time points, wherein said assessing is based at least on said identification or said indication of urologic condition determined in (c) at each of said plurality of time points.

53. (canceled)

54. The method of claim 1, wherein said urologic condition is selected from the group consisting of bladder cancer, kidney cancer, and prostate cancer.

55. (canceled)

56. The method of claim 55, wherein determining a quantitative measure indicative of said presence, absence, or relative assessment of bladder cancer of said subject comprises determining quantitative measures of one or more bladder cancer-associated genomic loci selected from the group consisting of TP53, KDM6A, MLL2, ARID1A, PIK3CA, RHOA, CDKN2A, PPARG, ATM, TP53, PTEN, BEND3, and PLEKHS1.

57-60. (canceled)

61. The method of claim 1, wherein said biological sample is a cell-free sample or a cell-associated sample.

62. A computer system for identifying or monitoring a urologic condition of a subject, comprising:
a database that is configured to store a dataset indicative of a presence, absence, or relative assessment of said urologic condition of said subject; and
one or more computer processors operatively coupled to said database, wherein said one or more computer processors are individually collectively programmed to:
(i) use a trained algorithm to process said dataset to determine a quantitative measure indicative of said presence, absence, or relative assessment of said urologic condition of said subject;
(ii) based at least in part on said quantitative measure, identify or provide an indication of said urologic condition of said subject with one or more of: (i) a sensitivity of at least about 90%, (ii) a specificity of at least about 90%, (iii) a positive predictive value of at least about 90%, and (iv) a negative predictive value of at least about 90%; and
(iii) electronically output a report that identifies or provides an indication of said urologic condition of said subject.

63-64. (canceled)

65. A non-transitory computer readable medium comprising machine-executable code that, upon execution by one or more computer processors, implements a method for identifying or monitoring urologic condition of a subject, said method comprising:
(a) obtaining a dataset indicative of a presence, absence, or relative assessment of said urologic condition;
(b) using a trained algorithm to process said dataset to determine a quantitative measure indicative of said presence, absence, or relative assessment of said urologic condition of said subject;
(c) based at least in part on said quantitative measure, identifying or providing an indication of said urologic condition of said subject with one or more of: (i) a sensitivity of at least about 90%, (ii) a specificity of at least about 90%, (iii) a positive predictive value of at least about 90%, and (iv) a negative predictive value of at least about 90%; and
(d) electronically outputting a report that identifies or provides an indication of said urologic condition of said subject.

66-75. (canceled)
